SEQUENCE LISTING (10 sequences)

SEQ ID NO: 1 - protein, 700 aa, D. melanogaster
MSGVDQMKIPGNTAKSVEERNIPWSQQVDEASYENLSSPTHDEVSGNDNSMQFQYPPFNFKENCNTPWNNMNKGRKANANHDSYRRHGHSIPNNTRRDEHQVNPWNSRKPAAQSFRNENDQPSTKLDNRTSDEAQNQEVVAAPKKTTWASIASQPAKLTSRAASTTSNSKKKGPGMPPPPMVPGKHNLDVNVWDLPNNKPPPVPSPPSPIDLGYSDLSSDFSISSNTAPPLGARAEKVHKDTENFLKYEHKGLGNNNNFSRAKVSPTGPVRRNLGPQQPVHHAAPRPATNAGPFGPPNARRHDGGPHPSRNSERSGNYSSFRGSEFESASKFEYRDENQSRPVEATSATEELPVDSQLVLDELKDKNNYNPKVLDLKKAGSARFFVIKSYSEDDIHRSIKYEIWCSTDHGNKRLDDAFKERHEEGGNIMLFFSVNGSGHFCGMAQMMTPVDYNSTSSVWSQDKWRGKFKVKWIYVKDVPNGTLRHIRLENNENKSVTNSRDTQEVPNDKGIEVLQILHSYNHSTSIFDDFFHYEKKQEEEVSSKRPPMHGPDGNNHAPAPLARSFNRQADDKDRDRDGRGGMSQKHFNSGGGAGFRNPGHRGGAGSGGSHNNYSEYRRGFDIHKSSSDYQFKDRDGPDFDRENDFGSHYGSNNRKNEYKHNNTNKARLKTRDRDFSTEQIKIGVRFKDTDKDKMRSNEYS

SEQ ID NO: 2 - protein, 559 aa, Homo sapiens
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 3 - protein, 579 aa, Homo sapiens
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 4 - protein, 585 aa, Homo sapiens
MSATSVDQRPKGQGNKVSVQNGSIHQKDAVNDDDFEPYLSSQTNQSNSYPPMSDPYMPSYYAPSIGFPYSLGEAAWSTAGDQPMPYLTTYGQMSNGEHHYIPDGVFSQPGALGNTPPFLGQHGFNFFPGNADFSTWGTSGSQGQSTQSSAYSSSYGYPPSSLGRAITDGQAGFGNDTLSKVPGISSIEQGMTGLKIGGDLTAAVTKTVGTALSSSGMTSIATNSVPPVSSAAPKPTSWAAIARKPAKPQPKLKPKGNVGIGGSAVPPPPIKHNMNIGTWDEKGSVVKAPPTQPVLPPQTIIQQPQPLIQPPPLVQSQLPQQQPQPPQPQQQQGPQPQAQPHQVQPQQQQLQNRWVAPRNRGAGFNQNNGAGSENFGLGVVPVSASPSSVEVHPVLEKLKAINNYNPKDFDWNLKNGRVFIIKSYSEDDIHRSIKYSIWCSTEHGNKRLDAAYRSLNGKGPLYLLFSVNGSGHFCGVAEMKSVVDYNAYAGVWSQDKWKGKFEVKWIFVKDVPNNQLRHIRLENNDNKPVTNSRDTQEVPLEKAKQVLKIIATFKHTTSIFDDFAHYEKRQEEEEAMRRERNRNKQ

SEQ ID NO: 5 - DNA, 3198 bp, Homo sapiens
gtcgccgccgccgccgccattggagtcgacgcctcctcagtgcgtccgcgtcccgggctcaccgccgctgccgcctcgccaggggcccgcgcgcccagcagccgccgccgccgcccggccggcgcccggggaattggcggcggggcccggggccgcgcgagctagggtgacaggcccggcctctaggggaggcccgagccggcgggcgccccggccccgcgtctagttgttcatgaagcatgtcggccaccagcgtggacacccagagaacaaaaggacaagataataaagtacaaaatggttcgttacatcagaaggatacagttcatgacaatgactttgagccctaccttactggacagtcaaatcagagtaacagttacccctcaatgagcgacccctacctgtccagctattacccgccgtccattggatttccttactccctcaatgaggctccgtggtctactgcaggggaccctccgattccatacctcaccacctacggacagctcagtaacggagaccatcattttatgcacgatgctgtttttgggcagcctgggggcctggggaacaacatctatcagcacaggttcaattttttccctgaaaaccctgcgttctcagcatgggggacaagtgggtctcaaggtcagcagacccagagctccgcgtatgggagcagctacacctaccccccgagctccctgggtggcacggtggttgatgggcagccaggctttcacagcgacaccctcagcaaggcccccgggatgaacagcctggagcagggcatggttggcctgaagattggggacgtcagctcctccgccgtcaagacggtgggctctgtcgtcagcagcgtggcactgactggtgtcctttctggcaacggtgggacaaatgtgaacatgccagtttcaaagccgacctcgtgggctgccattgccagcaagcctgcaaaaccacagcctaaaatgaaaacaaagagcgggcctgtcatggggggtgggctgccccctccacccataaagcataacatggacattggcacctgggataacaaggggcctgtgccgaaggccccagtcccccagcaggcaccctctccacaggctgccccacagccccagcaggtggctcagcctctcccagcacagcccccagctttggctcaaccgcagtatcagagccctcagcagccaccccagacccgctgggttgccccacgcaacagaaacgcggcgtttgggcagagcggaggggctggcagcgatagcaactctcctggaaacgtccagcctaattctgcccccagcgtcgaatcccaccccgtccttgaaaaactgaaggctgctcacagctacaacccgaaagagtttgagtggaatctgaaaagcgggcgtgtgttcatcatcaagagctactctgaggacgacatccaccgctccattaagtactccatctggtgtagcacagagcacggcaacaagcgcctggacagcgccttccgctgcatgagcagcaaggggcccgtctacctgctcttcagcgtcaatgggagtgggcatttttgtggggtggccgagatgaagtcccccgtggactacggcaccagtgccggggtctggtctcaggacaagtggaaggggaagtttgatgtccagtggatttttgttaaggatgtacccaataaccagctccggcacatcaggctggagaataacgacaacaaaccggtcacaaactcccgggacacccaggaggtgcccttagaaaaagccaagcaagtgctgaaaattatcagttcctacaagcacacaacctccatcttcgacgactttgctcactacgagaagcgccaggaggaggaggaggtggtgcgcaaggaacggcagagtcgaaacaaacaatgagggcgaaccagtttcttacatgttctaacgtttgactttgaaaacagtttaaaacacgtgtgcttggtcagctccagtgtgtcgtcccgtgcgggggttgagtgttgcatctttgcctttcttgtcgttgatttttgcccagatggatctgcatttatttgtactttttctatgtattataatcctgtagaagtcactaataaaggagtattttttttgtcagcttatcaatcagactgatctaatgtgaaatgtaagtatccttaaaaacaaagcatctattttggcagaaattgtgttcttaaattcagtcatttgatattctgtgagacttcatatttctcatccctttattgctttttagcaaacataagaaaccatgagtcattttgtcatttagagtattctgataaaatctcttgaaaatactgaaatcaaaaggttaatgattttttgttcattctgatttgtcattttattatctgttatcggtctaaagtgctaatttacccatttgatttttctgctagacagataacttttaatttttcaaatttggcagacactttttttttttttttgaaaatctttccttccagatctgttgcccactgaacagccacccgtccctcactgtcctggtgtccgattgggctggatggtgttggggcatgatgtgtggaggaactggaaggtgctttaggtctggttcagggtcgggcattctttgttgtttgcacatctttttaaattttacaccttttcttaagaattctaatgccgtcttaagtttttataccaataatgctgagctttaagtgtaggatctggtagtacagacagtgtgatggatgatgctgctggttgtaaatttcatcgtgtgtgtctaattttttttcctgttgaatgggtaaaaacaaaacaaaactttttttagaagatgaatttgctgtcatgttttgtggaatgagggaccgttgagctcactaccacctggagtttgagttgaagcatgaaaatggtgcccatgcctgacgctccagcgcctggatctgcacgtgcccttgtagaggatccttaccgtcctagagagcagacgctttctgaaaactacttgctccaaaagaccctctgagttaacgtttcagctgtatcattagacttgtatttagagcgtgtcacttcctctgaactgttactgcctgaatggagtcctggacgacattgggtttttcctctaggagaatacaagccttaataaacaatactatttagcaaa

SEQ ID NO: 6 - DNA, 2744 bp, Homo sapiens
agagcgtcgccgagtcggagccggagcctgagccgcgcgctgtgtctccgctgcgtccgccgaggcccccgagtgtcagggacaaaagcctccgcctgctcccgcagacggggctcatctgccgccgccgccgcgctgaggagagttcgccgccgtcgccgcccgtgaggatctgagagccatgtcggccagcagcctcttggagcagagaccaaaaggtcaaggaaacaaagtacaaaatggatctgtacatcaaaaggatggattaaacgatgatgattttgaaccttacttgagtccacaggcaaggcccaataatgcatatactgccatgtcagattcctacttacccagttactacagtccctccattggcttctcctattctttgggtgaagctgcttggtctacggggggtgacacagccatgccctacttaacttcttatggacagctgagcaacggagagccccacttcctaccagatgcaatgtttgggcaaccaggagccctaggtagcactccatttcttggtcagcatggttttaatttctttcccagtgggattgacttctcagcatggggaaataacagttctcagggacagtctactcagagctctggatatagtagcaattatgcttatgcacctagctccttaggtggagccatgattgatggacagtcagcttttgccaatgagaccctcaataaggctcctggcatgaatactatagaccaagggatggcagcactgaagttgggtagcacagaagttgcaagcaatgttccaaaagttgtaggttctgctgttggtagcgggtccattactagtaacatcgtggcttccaatagtttgcctccagccaccattgctcctccaaaaccagcatcttgggctgatattgctagcaagcctgcaaaacagcaacctaaactgaagaccaagaatggcattgcagggtcaagtcttccgccacccccgataaagcataacatggatattggaacttgggataacaagggtcccgttgcaaaagccccctcacaggctttggttcagaatataggtcagccaacccaggggtctcctcagcctgtaggtcagcaggctaacaatagcccaccagtggctcaggcatcagtagggcaacagacacagccattgcctccacctccaccacagcctgcccagctttcagtccagcaacaggcagctcagccaacccgctgggtagcacctcggaaccgtggcagtgggttcggtcataatggggtggatggtaatggagtaggacagtctcaggctggttctggatctactccttcagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaataaaaggcagttctacacagactgcagcaacggttgcatctgcatatcctaagaggaaaaaatgaccttcaagagaattaggacttttttcttaatttcactgacttcagagacgattgcagacttgcagtttaagtattggaatttcacaaaagacataggacttaactggaaaatgaaaaaaaaaagaaaaagaaaaaactaaacaaaaaatccctctaggtagtttaggtgaaaaatgtcccttttattttggctttggttgtgatttcagagcataatgctatgtttttttgtctttttactatgtttttcggatttttaagtccgtaagtgcatacagttttctctaatttttaaaccctttcctcctcccattttgacatttgcacttggagaacacttgagttgtgaaggttttgggcatccaccccagaaagtgggaatttgattttatccttccgaactggaagaacatttttatgaagaatttttgtctaggagaatataacagtgttacccaaggttgtgtctttaagggtggttcattttctctgaccttttgttactcaaagtaaagtactaggagtcctaagaaatgttctgttcttgtacattatactgattaagtcaggattaatttgatttcaaagctgagaacagtggtaaaaactcgtttacagaaatgcattttggaagagaaaaatactgtaaaacgtgtcgtgaatgtttcttcagtttcttgttcagccaatgaggaaagggcattgcctttctttttaccattaatcacttctcaataaacgtgagatcctgttgagcatca

SEQ ID NO: 7 - DNA, 5095 bp, Homo sapiens
gagactagggagtctgtccgccattgtggacccgagaagcagagagcgagagggggaagaggagcgtgcaagcggaaaagacgggcctcttcctccgactcccgagcgcgaggccctcattttgggttctcagcgaacggcggcagcggcggcggctggaacaatcactcggccaagggcgacagccaactgctgtgagtgcacggggagaggcccaggcagcggcggcggcggcggctctcgggttgcggtgaagaatgtcagccactagcgtggatcagagacctaaagggcaaggaaataaagtttcagtacaaaacggttcgattcatcaaaaagatgctgtaaatgatgatgattttgagccatacttaagtagccagacaaatcagagtaacagctatccaccaatgtcagatccatacatgcctagttactatgctccatccattggatttccatattctcttggggaagcagcgtggtccacagctggagaccagcctatgccatatctgacaacctatggacaaatgagtaatggagaacatcactatataccagatggtgtatttagtcaacctggggcattaggaaatacccctccatttcttggtcaacatggatttaacttttttcctggtaatgctgatttctctacatgggggacaagtggatctcagggacaatcaacacaaagttctgcttatagtagcagttatggctatccacctagttctcttgggagagctattactgatggacaggctggatttggcaatgatactttgagtaaggtgcctggcattagcagtattgagcaaggcatgactggactgaaaattggtggtgacctgacagctgcagtgacaaaaactgtaggtacagctttgagcagcagtggtatgactagcattgcaaccaatagtgtgcccccagttagcagtgcagcacctaaaccaacctcctgggctgccattgccagaaagcctgccaaacctcaaccgaaacttaaacccaagggcaatgtgggaattgggggttctgctgtaccaccacctcctataaaacacaacatgaatattggaacttgggatgaaaaagggtcagtggtaaaggctccaccaacccaaccagttctgcctcctcaaactataatccagcagcctcagccattaattcaaccaccaccattggtgcaaagccaactgcctcaacagcagcctcaaccaccacaaccacagcagcaacaaggacctcagccacaggcccagcctcaccaagtgcagcctcaacagcagcagctgcagaatcgctgggtagctcctcgtaacaggggagcaggcttcaaccagaacaatggagcgggcagtgaaaactttggtttaggtgttgtacctgtcagtgcttcaccttctagtgtagaagtgcatcccgtgctggaaaagctaaaggccataaacaactataatcccaaagactttgattggaatctgaagaatggacgtgtgtttataattaaaagctactctgaggatgacatacatcgttccattaaatactctatctggtgtagtactgagcatggtaataagcgtttggatgcagcttaccgttccctgaatgggaaaggcccactctatttactcttcagtgtgaatggcagtggacatttttgtggagtggctgaaatgaagtctgttgtggactataatgcgtatgctggtgtctggtctcaggataagtggaagggcaaatttgaagttaaatggatctttgtcaaagatgttcccaataaccaattacggcatattcgcttagaaaataatgacaacaaaccggttaccaattcaagggacactcaagaggtacccctagaaaaagctaagcaagtgcttaaaataattgctactttcaagcataccacctcaatctttgatgactttgcacattatgaaaagcgtcaagaagaggaggaagccatgcgtagggagagaaatagaaacaaacaataaccgtatgaagatgtcctgttaaatttacaacactaacgatgtagactctggaaatgcctaataagtcaaagaagacgtattaaagctcttttctgcttaaggtgacatctttgaacactttaacacaaagttgactcttctcgtaatggttttcatcagcgcatctgcccttatactcttcaccaaacacacttgagaactgtaacttcgtcaagcactttctgtcctgaagcttttaccagtatctgctgtcttttgtaattatgcatcctagctaaggcacagaagactgaatgaatgcaaggattcattaactctttgaatttgttaaatactaacagttaaccattagaagtggttcaatgatgtaagagtcacactgcttcaactttttctttgttgtagtttttaaattgtcgatttttagctatttgacagattaaaagcaaaataatcatgccatatttagtcctggagttcaagtctaaatgttgatgtgaaaaattattgtagtaaacttttaatatggcaaagcaaccttaagctctattttagccaaatgaaacataatctgaaattatattagaacatttcccttgtcttcaaactgtttggtgtaacagaatattgatatgcagcttggtggatttcaccagttaatgcacattcttcttccctcctccccccattaatatgtatactgaaaaatgtgcatttgtctgaggaattattttgtttgctaccacttaatgaatctcaaaattttgagtaaatgtacctcagtctaatcagactttttatgacctttataactacatttaaaacccttaattcctatttctgggtgtttgcgagcctgattgctatcatgaagtaaaaatttattactctaggtattcactagctaaataaacatagttcttgtttagcaagcatatgttgttcctcagctcttttctccagcttttgcagtgtcctggcatccttaaaatactttgaaaatatggccttgatccatggattaaatcagtatctaagtgaatgtgttgatgttttattgatcagatctatataagtgggaatacagcatatatctggatattcttatagttatctttttaacatcttatttttttcattaattacatatcaacattaattttgtatcttgaagcaaattgattttgtataattaaatgtgtcaagcatctgtattaattgatttgatggcataaggttatgaaaataatgtactgccccatgtattactgttccaaaaggagaaagctatgtagaaagatacattaagggtgaaaatagcaatacagtagatttgaataccttgatgttttgcattacttcatttatgtttacatcatgtttagaaatgttttcatttactgtggtctttggtcacttcagctcaaagacctagtgatggatatttctttgaggctttcatttatataattttattttgtacaatgttttttttaaatgtgcaaatactgtattcaagtgaaaaaaatacagtatttgtagataaccatagctactacacagttcttcggtagtcccagtgtagttatatcagtgtttactgaagggaacatcaaaatattaatggtatattataaaataaagactttcttaaaggaaaattgcacctattttacctttttaagagtaagccatgaaatcttgtaacatgtctcttaactatttataatgaaaagtggcatttgggtatagtcaccacagcaatgttctacatccctaagattatctaggtaggacatgtcaaagatgactgttgtcattctggaggtcctattagagaatattataaaagggtgaccttgtaggaaggatctgagtcctccccctgaggttctctttttcttggtgctttattagcaactctggatatttttataaaactagttacattataaacggtttcaaacatgtttaatttacattaggtttttatgtaagagtgtcatggaagcactcagcaagcaggctgattgcaatagactcagacatgcgaataaatgtaattgagagtctattcatggtgaggagtacatcccagtgcctttaacctggatttctaatcttaagtgaaatgggtgcagcattcctttggaaaaaaaaatctttttattttcaagtgataattttgtgtttttctcatataagttttctccagagcacccaccttctcttccttcttggtctgtcattatattgcaaaatatttttcctctgaatgaaattatcacaggttgtctcaagcacaaccaactgaatgtctcttaactgtggggaccaaaagggagagagcctggggtctacaagaggagacacatcatcaaatgtttgaatgatcacaaattaagacattatcagcccagtaaatttcttgcttaatgtttttccaagttctggcttgaatatttcttattaaagctatcttatgtgggtattttattttgaaaggtattatagtttgtatatttaacagtaaggaggaaactgtaaccaaaattagtatttctctatacgtattggtacttgaagattcctttcaaaagaaatccagcgttttcctaattttagtacttaatttctctttttaatttaagtgatctttctaattcgaaagctgtgttctttttgaataccgtgcatgggggttaagctgatgttaaaacagtttgcaataaaaaaaaatgaatcagcttaagtcatttaatcatttcaagtgcattctgcatcctttaaaaataagtttaagaaatttaagagaattgtgttttcattaagttttgcatatcttttgttatgccatgtaaattccctttttcgtatgattaaaggaaggttatgataaaatgattagttcatttacattcacttgtagcaattacatgagaatttgaattttgtcgtgtttgggtttgttcattcctgtgaatgatggtacagttaggtgagattttctgttatggtacccaaactcaccatttggtcctctttaatctttgagggtttcaataaaaattgttcactca

SEQ ID NO: 8 - DNA, 1755 bp, Artificial Sequence
  Reverse translation of YTDHF1
augagcgcgaccagcguggaucagcgcccgaaaggccagggcaacaaagugagcgugcagaacggcagcauucaucagaaagaugcggugaacgaugaugauuuugaaccguaucugagcagccagaccaaccagagcaacagcuauccgccgaugagcgauccguauaugccgagcuauuaugcgccgagcauuggcuuuccguauagccugggcgaagcggcguggagcaccgcgggcgaucagccgaugccguaucugaccaccuauggccagaugagcaacggcgaacaucauuauauuccggauggcguguuuagccagccgggcgcgcugggcaacaccccgccguuucugggccagcauggcuuuaacuuuuuuccgggcaacgcggauuuuagcaccuggggcaccagcggcagccagggccagagcacccagagcagcgcguauagcagcagcuauggcuauccgccgagcagccugggccgcgcgauuaccgauggccaggcgggcuuuggcaacgauacccugagcaaagugccgggcauuagcagcauugaacagggcaugaccggccugaaaauuggcggcgaucugaccgcggcggugaccaaaaccgugggcaccgcgcugagcagcagcggcaugaccagcauugcgaccaacagcgugccgccggugagcagcgcggcgccgaaaccgaccagcugggcggcgauugcgcgcaaaccggcgaaaccgcagccgaaacugaaaccgaaaggcaacgugggcauuggcggcagcgcggugccgccgccgccgauuaaacauaacaugaacauuggcaccugggaugaaaaaggcagcguggugaaagcgccgccgacccagccggugcugccgccgcagaccauuauucagcagccgcagccgcugauucagccgccgccgcuggugcagagccagcugccgcagcagcagccgcagccgccgcagccgcagcagcagcagggcccgcagccgcaggcgcagccgcaucaggugcagccgcagcagcagcagcugcagaaccgcuggguggcgccgcgcaaccgcggcgcgggcuuuaaccagaacaacggcgcgggcagcgaaaacuuuggccugggcguggugccggugagcgcgagcccgagcagcguggaagugcauccggugcuggaaaaacugaaagcgauuaacaacuauaacccgaaagauuuugauuggaaccugaaaaacggccgcguguuuauuauuaaaagcuauagcgaagaugauauucaucgcagcauuaaauauagcauuuggugcagcaccgaacauggcaacaaacgccuggaugcggcguaucgcagccugaacggcaaaggcccgcuguaucugcuguuuagcgugaacggcagcggccauuuuugcggcguggcggaaaugaaaagcgugguggauuauaacgcguaugcgggcguguggagccaggauaaauggaaaggcaaauuugaagugaaauggauuuuugugaaagaugugccgaacaaccagcugcgccauauucgccuggaaaacaacgauaacaaaccggugaccaacagccgcgauacccaggaagugccgcuggaaaaagcgaaacaggugcugaaaauuauugcgaccuuuaaacauaccaccagcauuuuugaugauuuugcgcauuaugaaaaacgccaggaagaagaagaagcgaugcgccgcgaacgcaaccgcaacaaacag

SEQ ID NO: 9 - DNA, 1737 bp, Artificial Sequence
  Reverse translation of YTDHF2
augagcgcgagcagccugcuggaacagcgcccgaaaggccagggcaacaaagugcagaacggcagcgugcaucagaaagauggccugaacgaugaugauuuugaaccguaucugagcccgcaggcgcgcccgaacaacgcguauaccgcgaugagcgauagcuaucugccgagcuauuauagcccgagcauuggcuuuagcuauagccugggcgaagcggcguggagcaccggcggcgauaccgcgaugccguaucugaccagcuauggccagcugagcaacggcgaaccgcauuuucugccggaugcgauguuuggccagccgggcgcgcugggcagcaccccguuucugggccagcauggcuuuaacuuuuuuccgagcggcauugauuuuagcgcguggggcaacaacagcagccagggccagagcacccagagcagcggcuauagcagcaacuaugcguaugcgccgagcagccugggcggcgcgaugauugauggccagagcgcguuugcgaacgaaacccugaacaaagcgccgggcaugaacaccauugaucagggcauggcggcgcugaaacugggcagcaccgaaguggcgagcaacgugccgaaaguggugggcagcgcggugggcagcggcagcauuaccagcaacauuguggcgagcaacagccugccgccggcgaccauugcgccgccgaaaccggcgagcugggcggauauugcgagcaaaccggcgaaacagcagccgaaacugaaaaccaaaaacggcauugcgggcagcagccugccgccgccgccgauuaaacauaacauggauauuggcaccugggauaacaaaggcccgguggcgaaagcgccgagccaggcgcuggugcagaacauuggccagccgacccagggcagcccgcagccggugggccagcaggcgaacaacagcccgccgguggcgcaggcgagcgugggccagcagacccagccgcugccgccgccgccgccgcagccggcgcagcugagcgugcagcagcaggcggcgcagccgacccgcuggguggcgccgcgcaaccgcggcagcggcuuuggccauaacggcguggauggcaacggcgugggccagagccaggcgggcagcggcagcaccccgagcgaaccgcauccggugcuggaaaaacugcgcagcauuaacaacuauaacccgaaagauuuugauuggaaccugaaacauggccgcguguuuauuauuaaaagcuauagcgaagaugauauucaucgcagcauuaaauauaacauuuggugcagcaccgaacauggcaacaaacgccuggaugcggcguaucgcagcaugaacggcaaaggcccgguguaucugcuguuuagcgugaacggcagcggccauuuuugcggcguggcggaaaugaaaagcgcgguggauuauaacaccugcgcgggcguguggagccaggauaaauggaaaggccgcuuugaugugcgcuggauuuuugugaaagaugugccgaacagccagcugcgccauauucgccuggaaaacaacgaaaacaaaccggugaccaacagccgcgauacccaggaagugccgcuggaaaaagcgaaacaggugcugaaaauuauugcgagcuauaaacauaccaccagcauuuuugaugauuuuagccauuaugaaaaacgccaggaagaagaagaaagcgugaaaaaagaacgccagggccgcggcaaa

SEQ ID NO: 10 - DNA, 1755 bp, Artificial Sequence; identical to SEQ ID NO: 8
  Reverse translation of YTDHF3

(19) United States
(12) Patent Application Publication
(10) Pub. No.: US 2022/0090073 A1
(43) Pub. Date: Mar. 24, 2022

(54) METHODS AND COMPOSITIONS FOR THE TREATMENT OF NEURODEGENERATIVE DISEASES

(71) Applicant: Alexandra PERLEGOS, Fremont, CA (US)

(72) Inventor: Alexandra PERLEGOS, Fremont, CA (US)

(21) Appl. No.: 17/484,901

(22) Filed: Sep. 24, 2021

Related U.S. Application Data
(60) Provisional application No. 63/083,087, filed on Sep. 24, 2020.

(51) Int. Cl. C12N 15/113; C12N 15/86; A61K 31/711; A61K 31/7115

(52) U.S. Cl. CPC C12N 15/113; A61K 31/7115; A61K 31/711; C12N 15/86

(57) ABSTRACT

Recombinant constructs and cells, as well as methods, for modulating mRNA translation by targeting YTHDF proteins, which play a role in the recognition of m⁶A methylation of mRNA transcripts, are provided.

### CROSS-REFERENCE TO RELATED APPLICATION

This application claims priority to U.S. Provisional Application No. 63/083,087, filed on Sep. 24, 2020, the entire contents of which are hereby incorporated by reference.

### TECHNICAL FIELD

The disclosure provides methods for modulating protein expression levels in eukaryotic cells, e.g., to treat neurodegenerative diseases, as well as recombinant constructs and cell lines related to the same.

### BACKGROUND OF THE DISCLOSURE

The regulation of gene expression is critical for growth and development, as well as for the proper maintenance of homeostasis in the face of changing environmental conditions. As such, cells utilize a variety of mechanisms to increase or decrease the production of specific gene products (e.g., proteins or RNA). Expression levels may be modulated, e.g., to trigger developmental pathways, in response to environmental stimuli, or to adapt to new food sources. Gene expression may be modulated at the transcriptional level, e.g., by increasing or decreasing the rate of transcriptional initiation, or aspects of RNA processing. It may also be controlled at the post-translational modification of proteins (e.g., by increasing or decreasing the rate of degradation). A myriad of different mechanisms for controlling gene expression exist in nature, and these mechanisms are typically linked to form complex regulatory networks.

The use of different mechanisms and triggers permits cells to express specific subsets of genes, or to adjust the level of particular gene products, on an as-needed basis. Doing so conserves energy and resources while also allowing cells to respond more quickly to environmental stimuli. For example, bacteria and eukaryotic cells often adjust the expression of enzyme used in synthetic or metabolic pathways based upon the availability of required substrates or end products. Similarly, many cell types will induce synthesis of protective molecules (e.g., heat shock proteins) in response to environmental stress.

A variety of endogenous mechanisms for modulating gene expression have been identified in eukaryotes. For example, it is known that DNA can be reversibly modified by the addition of methyl groups at specific positions, and that such modifications can repress or enhance transcriptional activity (by causing chromatin to condense or relax, by recruiting enzymes involved in the transcription of mRNAs, etc.). As such, the methylation of DNA serves as a mechanism for controlling gene expression at the transcription stage. In recent years, research has revealed that an analogous pathway regulates the methylation of mRNA transcripts. For example, the 3′UTR, coding sequence, and 5′UTR of mRNA transcripts may be modified by N⁶-methylation of adenosine (m⁶A), and methylation of C⁵ of cytosine (m⁵C). See, e.g., Fu et al., “Gene expression regulation mediated through reversible m 6 A RNA methylation.” Nature Reviews Genetics 15.5 (2014): 293; Squires et al., “Widespread occurrence of 5-methylcytosine in human coding and non-coding RNA.” Nucleic Acids Research 40.11 (2012): 5023-5033; Yi and Pan, “Cellular dynamics of RNA modification.” Accounts of Chemical Research 44.12 (2011): 1380-1388. Recent studies suggest that m⁶A methylation is the most prevalent internal modification of eukaryotic mRNA. See Dominissini et al., “Topology of the Human and Mouse m6A RNA Methylomes Revealed by m⁶A-Seq,” Nature 485:201-206 (2012); Meyer et al., “Comprehensive Analysis of mRNA Methylation Reveals Enrichment in 3′ UTRs and Near Stop Codons,” Cell 149:1635-1646 (2012). The presence of m⁶A moieties on mRNA transcripts has been shown to modulate mRNA splicing, export, localization, translation, and stability. See Maity et al., “N6-methyladenosine modification in mRNA: machinery, function and implications for health and diseases.” The FEBS Journal 283.9 (2016): 1607-1630.

While it known that m⁶A methylation plays a role in various aspects of mRNA transport, metabolism, and translation, prior research has failed to produce therapeutic agents, constructs, and methods based on the modulation of m⁶A methylation reader proteins, for the treatment of human diseases and disorders.

### BRIEF SUMMARY OF EXEMPLARY EMBODIMENTS OF THE DISCLOSURE

The present disclosure addresses various needs in the art by providing recombinant constructs and cells, as well as methods, for modulating mRNA translation by targeting YTHDF proteins, which play a role in the recognition of m⁶A methylation of mRNA transcripts, as shall be explained in further detail herein. These methods may be used, e.g., in connection with methods of treating human neurodegenerative diseases and disorders, such as Alzheimer's disease (AD), amyotrophic lateral sclerosis (ALS), frontotemporal dementia (FTD), or Parkinson's disease (PD). In some aspects, the present methods are performed by administering a therapeutic agent that modulates the level of one or more YTHDF proteins in a human subject in need of treatment. In others, such methods may be performed by administering recombinant genetic constructs or cells designed to express one or more YTHDF proteins, or which modulate endogenous expression levels of YTHDF proteins (e.g., a viral vector configured to transfect one or more cells of the subject, which expresses a YTHDF protein).

In a first general aspect, the present disclosure provides a nucleic acid molecule encoding a human YTHDF protein (e.g., YTHDF1, YHTDF2, or YTHDF3), which may, e.g., be operably linked to a heterologous promoter and/or upstream activation sequence (UAS) or include one or more modified nucleosides, as described in further detail herein. In some aspects, the YTHDF protein has an amino acid sequence of SEQ ID NOs: 2, 3, or 4. In some aspects, the YTHDF protein has an amino acid sequence that is at least 90%, 95% or 98% identical to the sequence of SEQ ID NOs: 2, 3, or 4, wherein the YTHDF protein is capable of specifically binding to m⁶A-modified mRNA, when expressed in a human cell.

In another general aspect, the present disclosure provides a nucleic acid molecule encoding a fragment of a human YTHDF protein (e.g., YTHDF1, YHTDF2, or YTHDF3), which may, e.g., be operably linked to a heterologous promoter and/or upstream activation sequence (UAS) or include one or more modified nucleosides, as described in further detail herein. In some aspects, the YTHDF protein fragment has an amino acid sequence consisting of a portion of SEQ ID NOs: 2, 3, or 4. In some aspects, the YTHDF protein fragment has an amino acid sequence comprising the amino acids at positions 1-50, 1-100, 1-150, 1-200, 1-250, 1-300, 1-350, 1-400, 1-450, or 1-500, or 1-550 of a human YTHDF protein (e.g., YTHDF1, YHTDF2, or YTHDF3). In some aspects, the YTHDF protein fragment has an amino acid sequence comprising the amino acids at positions 300-550, 300-400, 300-500, 350-400, 350-450, 350-500, 375-525, or 400-500 of a human YTHDF protein (e.g., YTHDF1, YHTDF2, or YTHDF3). In some aspects, the YTHDF protein fragment is capable of specifically binding to m6A-modified mRNA, when expressed in a human cell. In some aspects, the YTHDF protein fragment has an amino acid sequence that is at least 90%, 95%, or 98% identical to the sequence of any of the foregoing YTHDF protein fragments, wherein the YTHDF protein is capable of specifically binding to m6A-modified mRNA, when expressed in a human cell.

Any of the nucleic acid molecules described herein may comprise a messenger RNA (mRNA). In some aspects, the nucleic acid molecule may comprise an mRNA that includes one or more 5′ and/or 3′ UTR elements flanking the coding sequence of the YTHDF protein or fragment. The 5′ and/or 3′ UTR elements may comprise elements which differ from the UTR elements present on endogenous human mRNA transcripts encoding YTHDF proteins. In some aspects, the nucleic acid molecule may comprise an mRNA that incorporates one or more modified nucleosides. For example, the mRNA may comprise one or more pseudouridine or 1-methylpseudouridine bases in place of any uracil bases present in an mRNA sequence encoding a YTHDF protein or fragment described herein.

In some aspects, the nucleic acid molecules described herein may be provided as a composition formulated for delivery to or transfection of a human subject. For example, the nucleic acid may be formulated as a composition wherein the nucleic acid is complexed with or encapsulated by a cationic lipid or polymer (e.g., lipid nanoparticles), such as TransIT-mRNA (Minis Bio LLC), or Lipofectamine (Invitrogen).

In some aspects, the disclosure provides recombinant YTHDF proteins, or fragments thereof, which may display an improved ability to cross the blood-brain barrier (BBB). For example, the nucleic acid molecules described herein may further encode an antibody fragment crystallizable (Fc) region. In some aspects, the antibody Fc region binds to a transferrin receptor.

In some aspects, the nucleic acid molecules described herein may be incorporated into: a) a plasmid; or b) a viral vector capable of transfecting a eukaryotic cell. For example, the viral vector may be an adenovirus, an adeno-associated virus, a retrovirus, and/or a lentivirus.

In some aspects, the nucleic acid molecules described herein may encode a YTHDF protein operably-linked to a cell or tissue-specific promoter, such as a neuron-specific promoter, or a glial cell-specific promoter.

In some aspects, the disclosure provides a recombinant eukaryotic cell, adapted to express any of the nucleic acid molecules described herein.

In another general aspects, the disclosure provides a method of treating a neurodegenerative disease or disorder in a subject in need thereof, comprising: a) administering a therapeutically-effective amount of a YTHDF protein comprising: i) YTHDF1, YTHDF2, or YTHDF3; or ii) a protein that is at least 95% identical to the sequence of SEQ ID NO: 2, 3, or 4, wherein the YTHDF protein is capable of specifically binding to m⁶A-modified mRNA, when expressed in a human cell; and b) reducing or eliminating at least one symptom of the neurodegenerative disease.

In some aspects, the neurodegenerative disease or disorder is Alzheimer's disease (AD), amyotrophic lateral sclerosis (ALS), frontotemporal dementia (FTD), or Parkinson's disease (PD).

In some aspects, a therapeutically-effective amount of the protein administered comprises an amount of the YTHDF protein that is sufficient to significantly induce a positive modification of a neurodegenerative disease or disorder described herein. In some aspects, a therapeutically-effective amount may be selected which is small enough to avoid serious side-effects.

In some aspects, the YTHDF protein is YTHDF1, YTHDF2, or YTHDF3, and a therapeutically-effective amount comprises an amount sufficient to increase the level of endogenous YTHDF1, YTHDF2, or YTHDF3, in a cell of the subject, by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100%. In some aspects, the level of protein is measured using a Western blot, 2D-PAGE, a spectroscopic method, or mass spectrometry. In some aspects, the cell is a neuron, a glial cell, an astrocyte, a microglial cell, or an oligodendrocyte.

In another general aspect, the disclosure provides a method of treating a neurodegenerative disease or disorder in a subject in need thereof, comprising: a) administering any of the nucleic acid molecules described herein to the subject, under conditions sufficient to cause expression of the encoded YTHDF protein or fragment in a cell of the central nervous system of the subject; and b) reducing or eliminating at least one symptom of the neurodegenerative disease or disorder.

In some aspects, the nucleic acid molecule is an mRNA molecule or a DNA molecule. In some aspects, administering the nucleic molecule to the subject comprises: administering a viral vector to the subject, wherein the viral vector encodes the nucleic acid molecule.

In some aspects, administering the nucleic molecule to the subject comprises: administering a formulation comprising an mRNA encoding a YTHDF protein or fragment described herein, complexed with or encapsulated by a cationic lipid or polymer (e.g., lipid nanoparticles), such as TransIT-mRNA (Mirus Bio LLC), or Lipofectamine (Invitrogen). In some aspects, the nucleic acid molecule may comprise an mRNA that incorporates one or more modified nucleosides. For example, the mRNA may comprise one or more pseudouridine or 1-methylpseudouridine bases in place of any uracil bases present in an mRNA sequence encoding any YTHDF protein or fragment described herein.

In some aspects, the YTHDF protein (e.g., YTHDF1, YTHDF2, or YTHDF3), or a fragment thereof, is expressed in a treated subject at a concentration that is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100% higher than the endogenous expression level of YTHDF1, YTHDF2, or YTHDF3, in the plasma, or in a cell, tissue, or organ of the central nervous system, of the subject. In some aspects, the cell is a neuron, a glial cell, an astrocyte, a microglial cell, or an oligodendrocyte.

Various exemplary aspects of the presently disclosed invention are set forth in the accompanying description below. Although methods and materials similar or equivalent to those described herein can be used in the practice or testing of the invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 is a diagram showing the components of the m⁶A methyltransferase complex.

FIG. 2 is a table showing the Drosophila homologs for selected human m⁶A complex proteins.

FIG. 3 is a phylogenetic tree showing the relationship between m⁶A complex proteins, e.g., YTHDF1, YTHDF2, and YTHDF3, and the Drosophila homolog Ythdf.

FIG. 4 is a diagram showing domain architecture schematics for the human YTHDF1, YTHDF2, and YTHDF3 proteins, and the Drosophila homolog Ythdf.

FIG. 5 is a graph summarizing the survival results observed when a population of D. melanogaster was engineered to express an RNAi construct targeting Ythdf in their neuron cells.

FIG. 6 is a graph summarizing the survival results observed when a population of D. melanogaster was engineered to express an RNAi construct targeting Ythdf in their glial cells.

FIG. 7 is a graph summarizing the survival results observed when a population of D. melanogaster was engineered to express TDP-43 and an RNAi construct targeting Ythdf in their neuron cells.

FIG. 8 is a graph summarizing the survival results observed when a population of D. melanogaster was engineered to express TDP-43 and either a) a control RNAi construct, b) an RNAi construct targeting Ythdf, or c) an RNA construct targeting Ythdc1, in all cells.

FIG. 9 is graph summarizing the survival results observed when a population of D. melanogaster was engineered to express TDP-43 and a construct encoding a) a control RNAi, or b) Ythdf operably-linked to an upstream activation sequence, in all cells.

FIG. 10 is a graph summarizing the survival results observed when a population of D. melanogaster was engineered to express a control RNAi construct or Ythdf, in their neuron cells, and subjected to a 1.5 hour heat shock at 38.5° C.

FIG. 11 shows cross-sectional views of the brain of four D. melanogaster specimens engineered to express a control RNAi construct or an RNAi construct targeting Ythdf, in their neurons. These cross-section views were obtained from flies at an age of 30 or 50 days.

FIG. 12 is a graph showing the quantified volume of brain vacuoles observed in multiple brains of control RNAi construct or an RNAi construct targeting Ythdf.

FIG. 13 is an annotated photograph of Western blots showing protein expression levels in D. melanogaster male heads (left) and brains (right) following Ythdf knock-down and a 24-hour puromycin treatment. Puromycin acts as an aminoacyl-tRNA analog, and is incorporated into nascent peptides causing termination, but also labeling newly synthesized proteins, whose levels are readily measured with an anti-puromycin antibody.

FIG. 14 is a graph quantifying the relative intensity of the protein bands detected in the Western blots shown in FIG. 13.

FIG. 15 is an annotated photograph of immunoblots showing the results of a brain puromycin incorporation assay in D. melanogaster specimens that were either subjected to RNA interference targeting Ythdf, or engineered to upregulate Ythdf by expressing UAS-Ythdf (ElavGS>Ythdf).

FIG. 16 is a graph showing the relative intensity of the protein bands detected in the immunoblots shown in FIG. 15.

FIG. 17 is an annotated photograph of a Western blot showing the relative expression levels of eif2α-phosphorylation and tubulin, in D. melanogaster populations engineered to express an RNAi construct targeting Ythdf or a control, in their neurons, with or without a heat shock treatment.

FIG. 18 is a graph summarizing the relative levels of eif2α-phosphorylation in the D. melanogaster populations identified in FIG. 17.

FIG. 19 is an annotated photograph of a Western blot showing protein expression levels in D. melanogaster specimens engineered to express TDP-43 and an RNAi construct targeting either a control protein or Ythdf, following a 24-hour puromycin treatment. Puromycin acts as an aminoacyl-tRNA analog, and is incorporated into nascent peptides causing termination, but also labeling newly synthesized proteins, whose levels are readily measured with an anti-puromycin antibody.

FIG. 20 is an annotated photograph of a Western blot showing protein expression levels of eif2α-phosphorylation and tubulin (a control) in D. melanogaster specimens engineered to express TDP-43 and an RNAi construct targeting either a control protein or Ythdf.

FIG. 21 is a graph showing the relative intensity of the protein bands detected in the Western blots shown in FIG. 20.

FIG. 22 is a graph showing the survival rate of D. melanogaster specimens engineered to upregulate Ythdf expression over a 60-day period.

### DETAILED DESCRIPTION

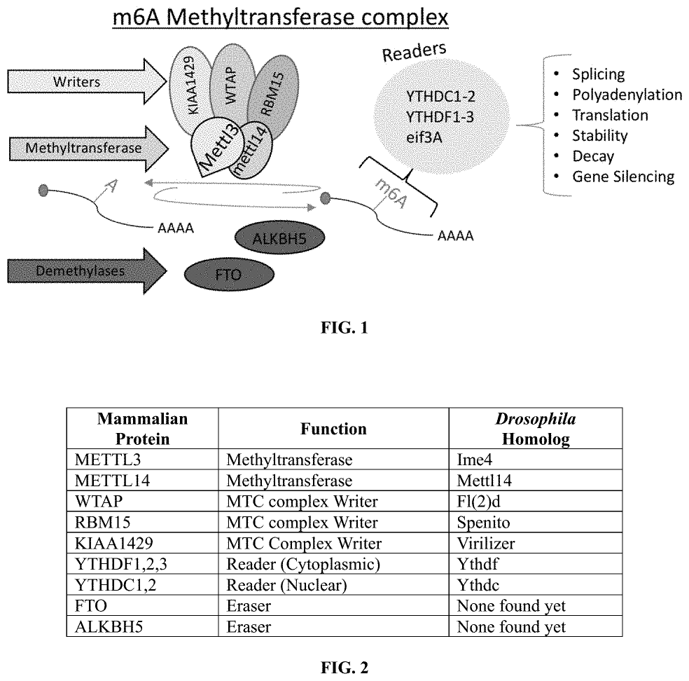

There exists a need in the art for therapeutic agents, constructs, and methods which target proteins involved in the m⁶A methylation pathway, for the treatment of human diseases and disorders (e.g., neurodegenerative diseases). The addition of m⁶A to mRNAs is mediated by a heterotrimeric protein complex consisting of the two methyltransferase-like (METTL) enzymes, METTL3 and METTL14, and their cofactor, Wilms tumor 1-associated protein (WTAP), which together function as m⁶A “writer” proteins. In mammals, M⁶A residues can be “erased” from mRNAs via oxidative demethylation by two known demethylases, FTO and ALKBH5. In general, m⁶A sites on mRNAs are “read” by three related YTH-domain containing family (“YTHDF”) proteins that localize to the cytoplasm of mammalian cells, i.e YTHDF 1, YTHDF2, and YTHDF3. The three YTHDF proteins all contain a conserved carboxy-terminal YTH domain that binds to m⁶A and a variable amino-terminal effector domain. In mammals, YTHDF1 facilitates the translation of m⁶A-modified mRNAs by interacting with initiation factors and facilitating ribosome loading, whereas YTHDF2 promotes the degradation of m⁶A-modified transcripts (e.g., by localizing m⁶A-modified mRNA to processing bodies in the cytoplasm). The function of YTHDF3 is less clear. However, YTHDF3 is thought to cooperate with YTHDF 1 and YTHDF2, enhancing the translation and decay of methylated mRNAs coordinated by YTHDF1 and YTHDF2. FIG. 1. shows a simplified representation of the primary enzymes involved in the m⁶A methylation pathway.

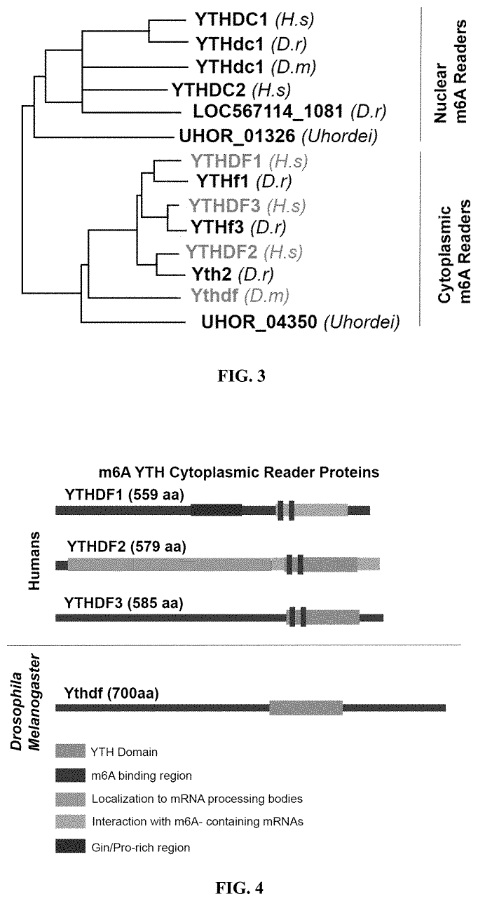

The m⁶A methylation pathway is widely conserved among eukaryotes, and has been documented in yeast, plants, flies, and mammals, as well as in viral RNAs which have a nuclear phase. For example, putative homologs for all of the major m⁶A methylation pathway enzymes exist in the fruit fly (Drosophila melanogaster), as shown by FIG. 2. With respect to “reader” proteins, sequential analysis reveals that the D. melanogaster genome encodes two YTH domain-containing proteins, i.e., the nuclear protein Ythdc1 and the cytoplasmic protein Ythdf. As illustrated by the phylogenetic tree shown in FIG. 3, further sequential analysis indicates that Ythdf is more closely related to the mammalian YTHDF proteins (i.e., YTHDF1, YTHDF2, and YTHDF3), whereas YT521-B is more closely related to YTHDC1 and YTHDC2.

FIG. 4 illustrates the domain architecture of the human YTHDF proteins YTHDF1, YTHDF2, and YTHDF3, and the D. melanogaster homolog Ythdf. In this case, the NCBI reference sequences for these proteins were used for the comparison, i.e., YTHDF1 comprises NCBI Reference Sequence No. NP_060268.2 (SEQ ID NO: 2, 559 aa), YTHDF2 comprises NCBI Reference Sequence No. NP_057342.2 (SEQ ID NO: 3, 579 aa), YTHDF3 comprises NCBI Reference Sequence No. NP_689971.4 (SEQ ID NO: 4, 585 aa), and their D. melanogaster homolog Ythdf comprises NCBI Reference Sequence No. NP_651322.1 (SEQ ID NO: 1, 700 aa). As used herein, references to the proteins YTHDF1, YTHDF2, YTHDF3, and Ythdf, should be understood as references to the respective NCBI reference sequences identified in this passage, unless otherwise noted. Similarly, references to nucleic acid molecules encoding the proteins YTHDF1, YTHDF2, YTHDF3, and Ythdf, should be understood as referring to any DNA or RNA nucleotide sequences that encode the respective NCBI reference sequences identified in this passage, according to the standard human codon table, unless otherwise noted. For example, in some aspects the disclosure contemplates specific nucleotide sequences for each of these proteins, e.g., the nucleotide sequences encoding YTHDF1, YTHDF2, YTHDF3, and Ythdf, may comprise the sequences represented by SEQ ID NOs: 5-8, respectively.

In some aspects, the present disclosure provides constructs or methods that utilize sequential variants of the proteins described herein. For example, sequential variants of YTHDF1, YTHDF2, and YTHDF3, sharing at least 80, 81, 82, 83, 84, 85, 86, 87, 88, 89, 90, 91, 92, 93, 94, 95, 96, 97, 98, or 99% sequence identity with any one of these proteins may be used in the constructs and methods described herein. In some aspects, the sequential variant will retain the capability to specifically binding to m⁶A-modified mRNA, when expressed in a human cell. With respect to nucleotide sequence, in some aspects sequential variants sharing at least 80, 81, 82, 83, 84, 85, 86, 87, 88, 89, 90, 91, 92, 93, 94, 95, 96, 97, 98, or 99% sequence identity with any one of SEQ ID Nos: 5-7 may be used in the constructs and methods described herein. For example, in some aspects the disclosure provides nucleic acid molecules comprising a codon-optimized sequence which encodes any one of YTHDF1, YTHDF2, or YTHDF3, wherein the codon-optimized sequence is configured to produce enhanced expression of the respective protein when expressed in a cell of a given organism. Optimized codon tables for various eukaryotes and bacteria are known in the art and compatible with such embodiments.

In some aspects, the disclosure also provides recombinant YTHDF proteins, e.g., variants of YTHDF1, YTHDF2, or YTHDF3, which comprise one or more mutations or truncations compared to the sequences encoded by SEQ ID NOs: 2-4, respectively. For example, mutations may comprise point mutations, the deletion of one or more amino acid residues, or the addition of one or more amino acid residues. For example, a variant of YTHDF1, YTHDF2, or YTHDF3 may be modified to include one or more protein tags used to purify or detect these proteins (e.g., a GST or His-tag, or a fluorescent tag such as GFP). Variant sequences may further comprise linker sequences (e.g., a short segment comprising a single amino acid).

In some aspects, the disclosure provides fusion proteins comprising an amino acid which includes the sequence of a YTHDF protein (or a variant thereof), operably linked to the amino acid sequence of one or more heterologous domains which provide additional functionality. Such fusion proteins may be used in any of the constructs, compositions, or methods described herein, in place of a YTHDF protein. In some aspects, a fusion protein may comprise the sequence of a YTHDF protein (or a variant thereof), operably linked to the amino acid sequence of an antibody or antibody fragment. Such fusion proteins may display an improved ability to cross the blood-brain barrier (BBB). For example, the nucleic acid molecules described herein may further encode an antibody Fc region operably linked to a sequence encoding a YTHDF protein (or variant thereof). In some aspects, the antibody Fc region binds to a transferrin receptor, or any other receptor expressed by a cell located at the BBB (e.g., endothelial cells of the capillary wall, astrocytes sheathing the capillary, and pericytes embedded in the capillary basement membrane.).

The YTHDF proteins described herein may be encoded by any nucleic acid molecule capable of being expressed and/or translated in a host cell. For example, a YTHDF protein may be encoded by DNA or mRNA. The DNA may be genomic DNA or non-genomic DNA (e.g., plasmid DNA). In some aspects, the disclosure provides mRNA encoding any of the YTHDF proteins described herein. Such constructs may be used, e.g., in methods of treatment by administering the mRNA to a subject as a therapeutic, resulting in translation of the YTHDF protein in at least one cell of the subject. Nucleic acid molecules encoding any of the YTHDF proteins described herein may comprise a heterologous or endogenous promoter, an internal ribosome entry site (IRES), and/or an enhancer operably-linked to the sequence encoding the YTHDF protein (e.g., to drive expression of the YTHDF protein).

As noted above, YTHDF proteins are known to function as “reader” proteins capable of detecting m⁶A methylation on mRNAs. Based on prior research, it is known that m⁶A methylation is enriched in mRNA transcripts isolated from mammalian brain cells, and that this mechanism plays a role in the regulation of cells and functions of the nervous system, e.g., in the self-renewal of neural stem cells, learning, memory, brain development, and synaptic growth. Despite these findings, there are no available therapeutic treatments for human neurodegenerative diseases and disorders based on the modulation of proteins involved in the m⁶A methylation pathway.

The present disclosure addressed this need by disclosing methods of treating neurodegenerative diseases and disorders such as AD, FTD, PD, and ALS, by modulating the expression level of one or more of the endogenous YTHDF proteins present in humans and other mammals (i.e., YTHDF1, YTHDF2, and YTHDF3), or by supplementing such level(s) (e.g., by administering YTHDF proteins, or constructs designed to express YTHDF proteins, to a subject in need thereof as a therapeutic).

As noted above, Ythdf is the D. melanogaster homolog of the YTHDF proteins found in humans and other mammals. In recent years, D. melanogaster has become widely recognized as a suitable animal model for human neurodegenerative diseases. See, e.g., McGurk, et al., “Drosophila as an in vivo model for human neurodegenerative disease,” Genetics 201.2 (2015): 377-402. As noted in this study by McGurk et al., the human protein known as TDP-43 (“Transactive Response (TAR) DNA binding protein of 43 kDa”) has been identified as a protein implicated in several neurodegenerative diseases, including ALS and FTD, and the expression of human TDP-43 in D. melanogaster causes pathological effects similar to those observed in ALS patients, e.g., neuronal degeneration, motor neuron deficits, and shortened lifespan. TDP-43 has also been implicated in AD, as described by Josephs, et al., “TDP-43 is a key player in the clinical features associated with Alzheimer's disease.” Acta neuropathologica 127.6 (2014): 811-824. TDP-43 is known to bind both DNA and RNA and has multiple functions in transcriptional repression, pre-mRNA splicing and translational regulation. However, as evidenced by these and other studies, TDP-43 overexpression, misfolding and/or mislocalization (e.g., due to mutation) is associated with the pathology of many neurodegenerative diseases.

The present disclosure is based in part on the surprising finding that Ythdf (i.e., the D. melanogaster homolog for the human YTHDF proteins), can be used to reduce the pathological effects observed when D. melanogaster is engineered to express human TDP-43. Moreover, as explained in further detail below, overexpression of a Ythdf protein in D. melanogaster (e.g., using an upstream activation sequence) rescues the reduced survival phenotype caused by TDP-43 expression, further demonstrating that YTHDF proteins can be used to modulate the pathological effects caused by TDP-43. In view of these and other findings disclosed herein, and the recognized association between TDP-43 and human neurodegenerative diseases, it is understood that YTHDF proteins (and sequential variants thereof) may be used as a therapeutic for human neurodegenerative diseases associated with TDP-43.

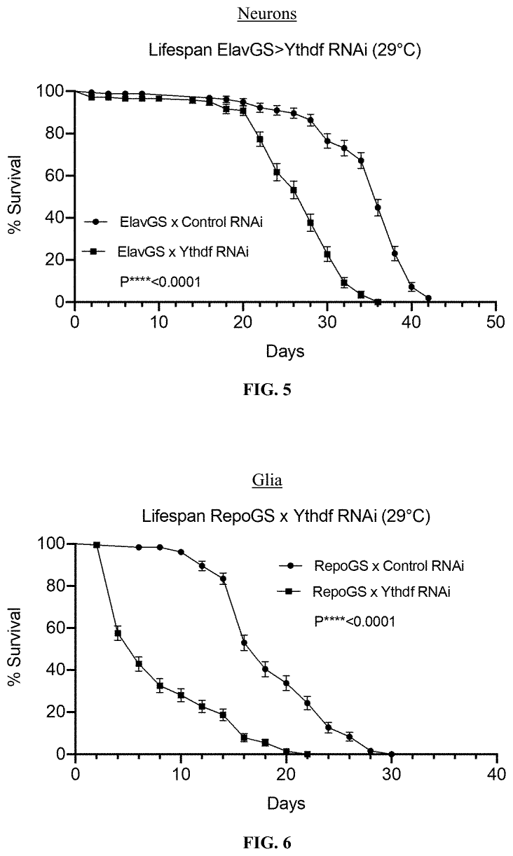

As demonstrated by FIGS. 5-6, Ythdf levels affect the average lifespan of D. melanogaster, with reduced levels leading to a reduction in lifespan. FIG. 5 shows the results of an experiment which examined the impact of Ythdf in a test population engineered to express an RNAi construct designed to knock-down levels of Ythdf, which was under the control of the Elav Gene Switch (“ElavGS”) system. The ElavGS system is a conditional tissue-specific transgene expression system that drives gene expression in larval neurons in an RU486-dependent manner. The ElavGS system is described in detail in Osterwalder et al., “A conditional tissue-specific transgene expression system using inducible GAL4.” Proceedings of the National Academy of Sciences 98.22 (2001): 12596-1260. As demonstrated by FIG. 5, Ythdf knock-down reduced the survival percentage of flies in the test group, with a noticeable drop beginning at day 20, compared to a control population engineered to express a non-functional RNAi construct using the ElavGS system (log-rank test p****<0.0001). FIG. 6 shows the results of a similar experiment, with the only modification being that the test population was engineered to use a different Gene Switch system, (i.e., the “RepoGS” system), which drives expression in glial cells, rather than neurons. In this experiment, the survival rate of treated flies dropped off precipitously by day 2, once again demonstrating the importance of YTHDF for survival (log-rank test p****<0.0001).

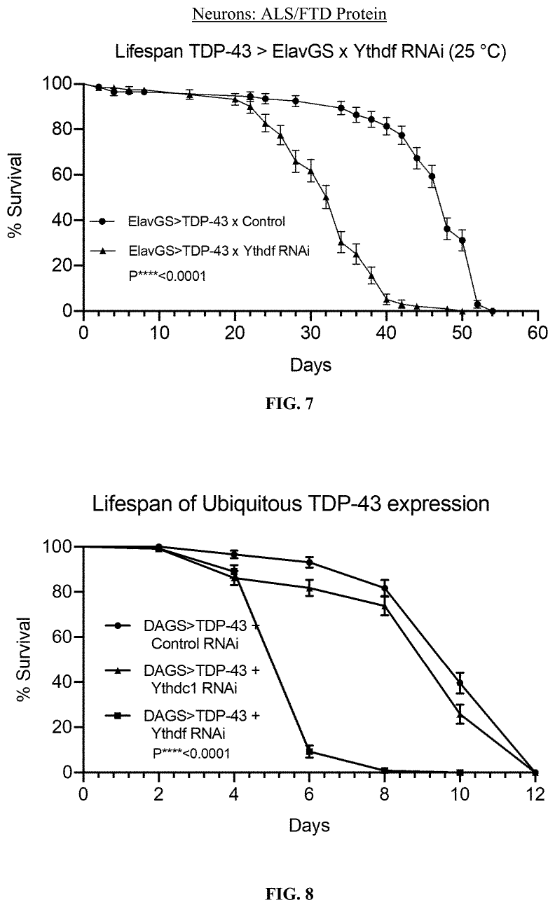

FIG. 7 shows the results of another experiment using the ElavGS system, wherein the control population was engineered to express human TDP-43 and the test population was engineered to express human TDP-43 plus an RNAi construct designed to knock-down expression of Ythdf, in neurons. TDP-43 is a human protein associated with several human neurodegenerative diseases, including AD and FTD. These results demonstrated by the test group, knock-down of Ythdf significantly reduced the survival rate of flies engineered to express TDP-43 (log-rank test p****<0.0001), further evidencing a functional relationship between these proteins.

As explained above, the genome of D. melanogaster encodes two YTH domain-containing proteins, Ythdf and Ythdc1. An experiment was conducted to determine whether Ythdc1 levels impact the survival rate of flies engineered to express TDP-43. For this experiment, the Daughterless Gene Switch (“DaGS”) system was used to drive expression of human TDP-43 and a control RNAi construct, or alternatively an RNAi construct designed to knock-down expression of Ythdf or Ythdc1, respectively. The DAGS system is designed to produce ubiquitous expression of a protein interest in all tissue types. As shown by FIG. 8, ubiquitous expression of human TDP-43 resulted in a substantial reduction in average lifespan, with a total loss of all treated flies by day 12. Moreover, the results of this experiment demonstrate that knock-down of Ythdc1 had little effect on the survival rate of treated flies, whereas knockdown of Ythdf once again led to a substantial decrease in the survival rate (log-rank test p****<0.0001).

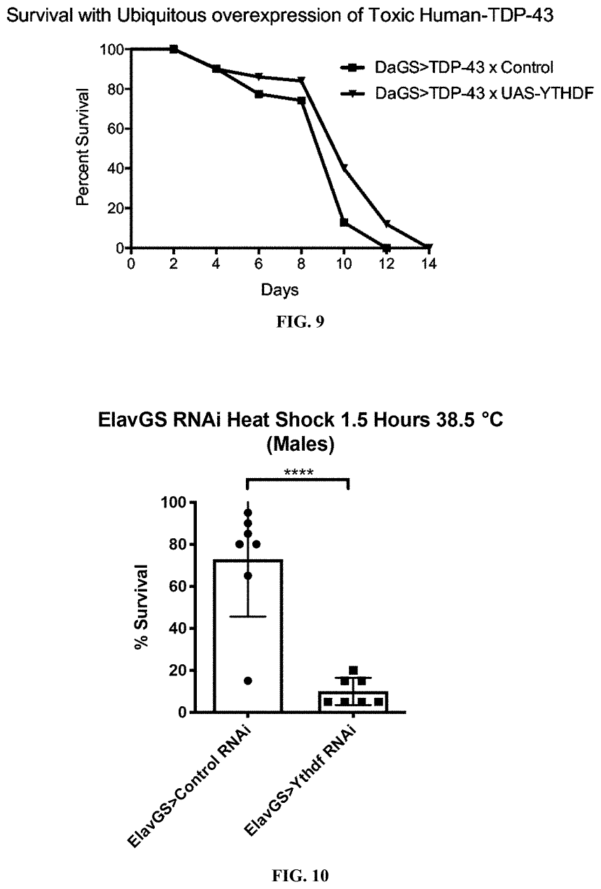

A subsequent experiment was conducted to determine whether overexpression of a Ythdf protein could rescue flies designed to express human TDP-43. For this study, the DaGS system was used to drive expression of human TDP-43 and either a control protein or a Ythdf protein operably linked to an upstream activation sequence. As shown by FIG. 9, co-expression of a Ythdf protein with TDP-43 resulted in an increase in survival, starting at day 4.

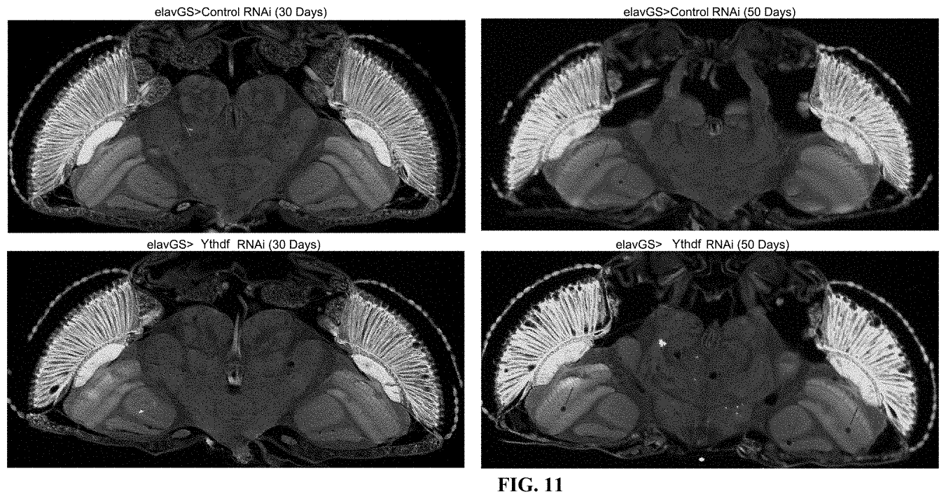

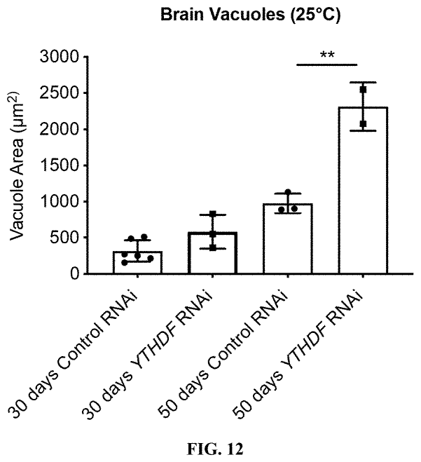

Further experiments demonstrate that the depletion of Ythdf in D. melanogaster results in a reduced ability to withstand stress (e.g., heat shock) and causes structural anomalies in the brain which resemble anomalies caused by human neurodegenerative diseases. To test the survival of Ythdf-deficient flies, the DaGS system was used to drive expression of an RNAi construct targeting Ythdf, in an RU486-dependent manner. The flies in this cohort were fed with food containing RU486 for 7 days post-eclosion and then subjected to a heat shock for 1.5 hours at 38.5° C. These flies were then placed back onto RU486 food and scored for survival 24 hours later. The results of this experiment are summarized by FIG. 10. Each point represents one vial of 20 flies and the survival rate observed for each vial. The survival rate for flies in this cohort remained high despite the reduced expression of Ythdf, up until the heat shock step. As shown by this graph, the survival rate for this cohort of flies dropped to approximately 10% survival following heat shock. Reduced levels of Ythdf, an m⁶a reader protein, may consequently hinder the stress response. In a related experiment, two groups of flies were engineered to express an RNAi construct targeting either a control protein, or Ythdf, in their neuron cells, and maintained at 25° C. for either 30 or 50 days. Paraffin sections of the brains of flies in both groups were prepared and analyzed to detect and measure the incidence of brain vacuoles. It is known in the art that fly brain vacuoles can be used to quantify neurodegeneration. See, e.g., Sunderhaus and Kretzschmar, “Mass histology to quantify neurodegeneration in Drosophila.” Journal of Visualized Experiments, 118 (2016): e54809. As shown by FIG. 11, flies in the experimental group (subject to Ythdf knock-down) displayed more brain vacuoles at both time-points compared to age matched control brains. The total size of brain vacuoles observed in both groups was quantified, as shown by FIG. 12. In brief, the area of brain vacuoles in 10 consecutive brain sections was measured. Each point on this graph represents the total area of vacuoles in 10 consecutive brain sections from one fly brain. A dramatic increase in total area of brain vacuoles in the experimental group is observed by day 50, evidencing increased neurodegeneration.

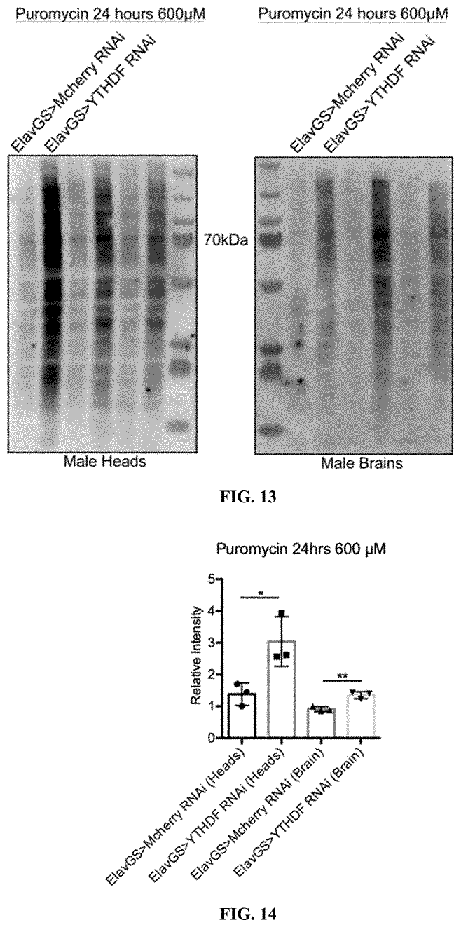

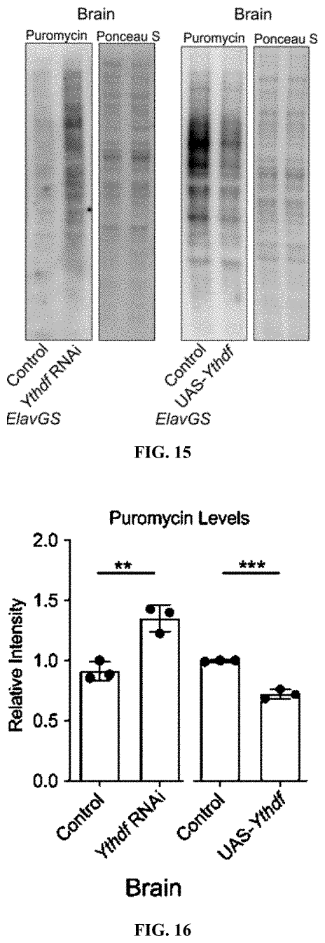

The impact of Ythdf-knockdown was further studied using a puromycin incorporation assay. As noted above, puromycin acts as an aminoacyl-tRNA analog, and is incorporated into nascent peptides causing termination, but also labeling newly synthesized proteins, whose levels are readily measured with an anti-puromycin antibody. In brief, flies were engineered to express an RNAi construct targeting either a control RNAi or Ythdf and placed on sucrose and agar food with 600 μM puromycin mixed in. Flies were allowed to eat this food for 24 hours. After 24 hours fly heads and brains were collected for Western blot samples. FIG. 13 shows three independent biological replicates from the heads and brains of flies. As illustrated by these Western blots, knock-down of Ythdf in fly neurons causes an increased amount of puromycin incorporation. This indicates an increased amount of protein translation at baseline in these flies. Relative protein expression levels for each of these Western blots was quantified, and is summarized by the graph shown in FIG. 14. This graph further confirms that there is a significant increase in puromycin incorporation in the cohort subjected to Ythdf-knockdown. Conversely, upregulation of Ythdf in neurons showed the opposite response, with significant decrease in nascent protein translation in dissected brains protein samples FIGS. 15 and 16.

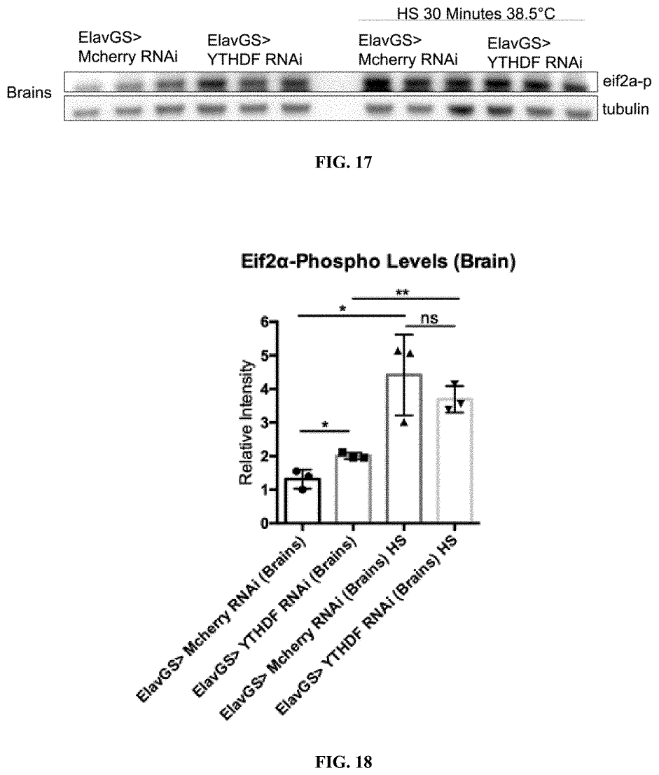

A similar experiment was conducted to evaluate whether Ythdf-knockdown has an impact on the level of eif2α-phosphorylation. In general, an increased level of phosphorylation of eif2a indicates increased levels of stress granules and cellular stress. Increased eif2a phosphorylation is further known to be associated with human neurodegenerative diseases such as AD. See, e.g., Ohno. “Roles of eIF2a kinases in the pathogenesis of Alzheimer's disease.” Frontiers in Molecular Neuroscience 7 (2014): 22. In brief, flies were engineered to express an RNAi control construct or a construct targeting Ythdf. Brains from flies in both groups were collected at baseline or after a 20-minute heat stress, and subjected to Western blotting with an antibody specific for phosphorylated eif2a to determine the level of phosphorylation of this translation initiation factor. As illustrated by the Western blot images shown in FIG. 17, and the graph shown in FIG. 18 (quantifying this data), at baseline there is an increased eif2α-phosphorylation compared to control flies, indicative of canonical translational repression. However, this pattern is not observed following heat shock. Overall, these results suggest that Ythdf plays a role in translation and the regulation of mRNA stability which may affect the overall cellular stress response.

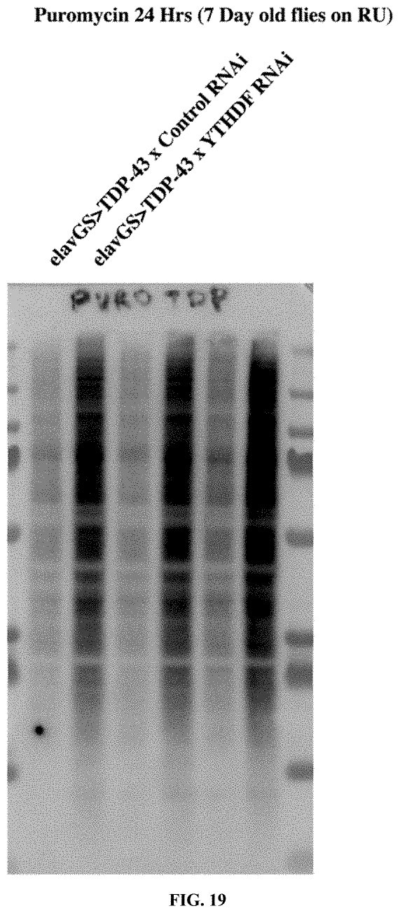

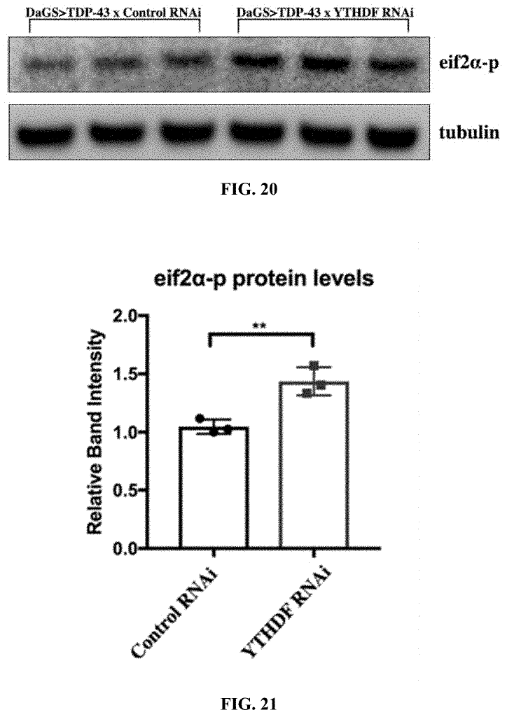

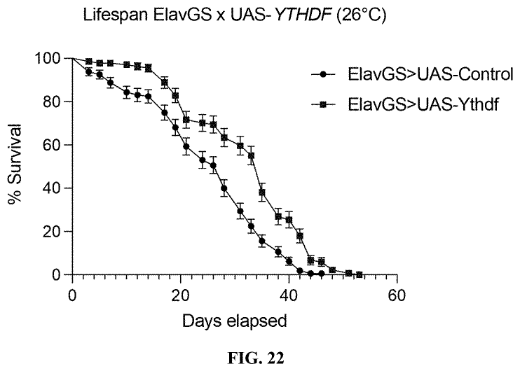

A similar experiment was conduct to evaluate the impact of Ythdf-knockdown on flies engineered to express human TDP-43. First, flies were engineered to express TDP-43, and an RNAi control construct or a construct targeting Ythdf, in their neurons using the ElavGS system. Both groups were then subjected to a puromycin incorporation assay (as described above). The heads of flies in both groups were then collected and used to prepare a Western blot using anti-puromycin antibodies. The results of this assay are shown in FIG. 19, and illustrate that a substantial increase in puromycin was observed in fly heads collected from the Ythdf-knockdown cohort after 24 hours on food containing puromycin. A follow-up study was conducted using flies engineered to express these same RNAi constructs, but under the control of the DaGS system (which provides ubiquitous expression). After knock-down, fly heads were collected from both groups and used as samples to prepare a second Western blot, which was probed with antibodies against phosphorylated eif2a and tubulin (used as a loading control). Images of this Western blot, and a graph showing quantitative protein level data, are provided in FIGS. 20 and 21, respectively. Both figures show that the level of eif2α-phosphorylation in fly heads collected from the Ythdf-knockdown group is increased compared to the control group. As explained above, TDP-43 has been identified as a protein implicated in several neurodegenerative diseases, including ALS and FTD, and the expression of TDP-43 in D. melanogaster causes pathological effects similar to those observed in ALS patients, e.g., neuronal degeneration, motor neuron deficits, and shortened lifespan. Furthermore, elevated levels of eif2α-phosphorylation are associated with TDP-43 and also with human neurodegenerative diseases generally. These experiments demonstrates that Ythdf is able to reduce the level of eif2α-phosphorylation. This set of data further suggests that Ythdf may be administered to a fly to rescue the neurodegeneration phenotype caused by TDP-43.

Based on these findings, and the similarities between Drosophila's Ythdf and the homologous human YTHDF proteins (i.e., YTHDF1, YTHDF2, and YTHDF3), it follows that human neurodegenerative diseases associated with elevated eif2α-phosphorylation levels may be treated by the administration of a YTHDF protein or by a nucleic acid construct designed to express a YTHDF protein (e.g., by the administration of one or more mRNA constructs encoding any of the YTHDF proteins or fragments described herein). For example, a method of treating a neurodegenerative disease (e.g., AD, ALS, PD) may comprise administering an effective amount of a YTHDF protein or fragment, or a nucleic acid construct designed to express the same, in accordance with the present disclosure, to a human subject in need thereof. The construct may be an mRNA, with or without chemical modifications such as the incorporation of one or more pseudouridine or 1-methylpseudouridine bases in place of any uracil bases present in an mRNA sequence encoding a YTHDF protein or fragment described herein. In some aspects, such treatment may reduce or eliminate one or more symptoms of the neurodegenerative disease, or delay onset or progression of the neurodegenerative disease.

In some aspects, a therapeutically-effective amount of the YTHDF protein may comprise an amount of YTHDF1, YTHDF2, or YTHDF3, or an mRNA construct encoding any of these YTHDF proteins, sufficient to increase the concentration of YTHDF1, YTHDF2, or YTHDF3, in the plasma, or in a cell, tissue, or organ of the subject, by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100% compared to the respective endogenous level. In other aspects, the amount may be sufficient to increase the level of endogenous YTHDF1, YTHDF2, or YTHDF3, to a level within a range bounded by a pair of endpoints selected from any of the levels listed in this passage (e.g., by 20-40% or by 60-100%). In some aspects, a therapeutically-effective amount of the YTHDF protein fragment may comprise an amount of any portion of the amino acid sequence of YTHDF1, YTHDF2, or YTHDF3. For example, in some aspects, the YTHDF protein fragment has an amino acid sequence comprising the amino acids at positions 300-550, 300-400, 300-500, 350-400, 350-450, 350-500, 375-525, or 400-500 of a human YTHDF protein (e.g., YTHDF1, YHTDF2, or YTHDF3). In some aspects, the YTHDF protein fragment is capable of specifically binding to m6A-modified mRNA, when expressed in a human cell. In some aspects, the YTHDF protein fragment has an amino acid sequence that is at least 90%, 95%, or 98% identical to the sequence of any of the foregoing YTHDF protein fragments, wherein the YTHDF protein is capable of specifically binding to m6A-modified mRNA, when expressed in a human cell.

In some aspects, the mRNA encoding a YTDHF1 protein or fragment thereof, according to any of the exemplary aspects described herein, may comprise the sequence of NCBI Reference Sequence Nos. NM_017798.4 (SEQ ID NO:5), NM_016258.3 (SEQ ID NO:6), or NM_152758.6 (SEQ ID NO:7), which encode human YTHDF1, 2, and 3, respectively. It is understood that these NCBI Reference Sequences show thymine (“T”) bases in place of the uracil (“U”) bases that would be present in an mRNA and that the mRNA sequences contemplated herein will include this substitution. In some aspects, the mRNA may encode a fragment of a YTHDF protein (e.g., a portion of the nucleotide sequences of any of SEQ ID NOs:5-7). For example, the mRNA may comprise the sequence of any one of SEQ ID NOs:5-7 with one or more intron sequence removed, or with an alternative 5′ or 3′ UTR. In some aspects, the mRNA may comprise a sequence that shares at least 90, 91, 92, 93, 94, 95, 96, 97, 98, 99, or 100% sequence identity with any one of SEQ ID NOs:5-7, or a portion thereof (e.g., a portion of the mRNA sequence encoding amino acids at positions 1-50, 1-100, 1-150, 1-200, 1-250, 1-300, 1-350, 1-400, 1-450, or 1-500, 1-550, 300-550, 300-400, 300-500, 350-400, 350-450, 350-500, 375-525, or 400-500 of YTHDF1, YHTDF2, or YTHDF3). In some aspects, the mRNA may comprise the sequence of any one of SEQ ID NOs: 8-10, which are reverse translations of human YTHDF1, YTHDF2, and YTHDF3, respectively. In some aspects, the mRNA may comprise a sequence that shares at least 90, 91, 92, 93, 94, 95, 96, 97, 98, 99, or 100% sequence identity with any one of SEQ ID NOs:8-10, or a portion thereof (e.g., a portion of the mRNA sequence encoding amino acids at positions 1-50, 1-100, 1-150, 1-200, 1-250, 1-300, 1-350, 1-400, 1-450, or 1-500, 1-550, 300-550, 300-400, 300-500, 350-400, 350-450, 350-500, 375-525, or 400-500 of YTHDF1, YHTDF2, or YTHDF3). In some aspects, the mRNA encoding the YTHDF protein or fragment has been codon optimized for expression in a human.

In some aspects, a nucleic acid construct (e.g., an mRNA) configured to express a YTHDF protein or fragment thereof, is administered to a human subject in an amount sufficient to cause a detectable expression of the YTHDF protein or fragment thereof, in a cell, tissue, or organ of the subject. Such constructs may comprise, e.g., a 5′ UTR, a codon optimized open reading frame encoding the polypeptide, a 3′ UTR and/or a 3′ tailing region of linked nucleosides. In some aspects, the nucleic acid construct may comprise a modified mRNA. For example, the mRNA may comprise pseudouridine (ψ), pseudouridine (w) and 5-methyl-cytidine (m⁵C), 1-methyl-pseudouridine (m¹ψ), 1-methyl-pseudouridine (m¹ψ) and 5-methyl-cytidine (m⁵C), 2-thiouridine (s²U), 2-thiouridine and 5-methyl-cytidine (m⁵C), 5-methoxy-uridine (mo⁵U), 5-methoxy-uridine (mo⁵U) and 5-methyl-cytidine (m⁵C), 2′-O-methyl uridine, 2′-O-methyl uridine and 5-methyl-cytidine (m⁵C), N6-methyl-adenosine (m⁶A) or N6-methyl-adenosine (m⁶A) and 5-methyl-cytidine (m⁵C). In other aspects, the mRNA may comprise pseudouridine (w), N1-methylpseudouridine (m¹ψ), 2-thiouridine, 4′-thiouridine, 5-methyl cytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine, or combinations thereof. In yet another aspect, the mRNA may comprise 1-methyl-pseudouridine (m¹ψ), 5-methoxy-uridine (mo⁵U), 5-methyl-cytidine (m⁵C), pseudouridine (ω), α-thio-guanosine, or α-thio-adenosine, or combinations thereof. In some aspects, the mRNA comprises pseudouridine or a pseudouridine analog. In some aspects, the mRNA comprises N1-methylpseudouridine. In some aspects, each mRNA comprises fully modified N1-methylpseudouridine. It is understood that these modified nucleosides may be incorporated in place of any of the corresponding standard analog nucleoside (e.g., pseudouridine in place of any uracil, 5-methylcytosine in place of any cytosine) in an mRNA sequence encoding any YTHDF protein or fragment described herein.

In some aspects, the mRNA encoding the YTHDF protein or fragment thereof may be administered to a human subject (e.g., in a therapeutic composition described herein) in an amount of 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, 300-400 μg, 350-450 μg, or 400-500 μg per dose. In some aspects, the dosage amount may comprise an amount that is less than, greater than, or within a range with endpoints defined by, any of the foregoing values. In some aspects, the mRNA is formulated as a therapeutic composition administered to the subject by intradermal or intramuscular injection. In some aspects, the therapeutic composition is formulated as a 0.5, 1.0, 2.0, 3.0, 4.0, 5.0, 6.0, 7.0, 8.0, 9.0, or 10.0 mL solution, or as a solution with a volume within a range with endpoints defined by any of the foregoing values. In some aspects, a therapeutic composition may comprise an mRNA encoding a YTHDF protein or fragment thereof, plus a carrier such as a lipid nanoparticle (LNP), and optionally one or more additional solvents, stabilizers, buffers, and/or excipients.

In some aspects, an LNP may comprise one or more lipids (e.g., one or more ionizable lipids, structural lipids, and/or phospholipids), and one or more mRNAs. The LNPs described herein may be used as a therapeutic composition for delivery of the mRNA constructs described herein. In some aspects, an LNP comprises an ionizable lipid, a structural lipid, a phospholipid, a PEG-modified lipid and one or more mRNAs. In some aspects, the LNP comprises an ionizable lipid, a PEG-modified lipid, a sterol and a phospholipid. In some aspects, the LNP has a molar ratio of about 20-60% ionizable lipid:about 5-25% phospholipid:about 25-55% sterol; and about 0.5-15% PEG-modified lipid. In some aspects, the LNP comprises a molar ratio of about 50% ionizable lipid, about 1.5% PEG-modified lipid, about 38.5% cholesterol and about 10% phospholipid. In some aspects, the LNP comprises a molar ratio of about 55% ionizable lipid, about 2.5% PEG lipid, about 32.5% cholesterol and about 10% phospholipid. In some aspects, the ionizable lipid is an ionizable amino or cationic lipid and the neutral lipid is a phospholipid, and the sterol is a cholesterol. In some aspects, the LNP has a molar ratio of 50:38.5:10:1.5 of ionizable lipid:cholesterol:DSPC (1,2-dioctadecanoyl-sn-glycero-3-phosphocholine):PEG-DMG.

In some aspects, a therapeutic composition comprising a YTHDF protein or fragment described herein, or one or more of the mRNA constructs described herein, is administered to a human subject at least once every 1, 2, 3, or 4 weeks, or at least once or twice per month, e.g., for the treatment of a neurodegenerative disease or disorder such as Alzheimer's disease (AD), amyotrophic lateral sclerosis (ALS), frontotemporal dementia (FTD), or Parkinson's disease (PD). Treatment may comprise a reduction of symptoms and/or a delay in the progression of any of the foregoing diseases or disorders.

In some aspects, any of the nucleotide constructs or YTHDF proteins described herein may be administered to a human subject in order to increase or improve longevity. As illustrated by the results shown in FIG. 22, upregulation of Ythdf has been demonstrated to improve the survival rate of D. melanogaster as compared to otherwise identical control samples. In some aspects, the constructs or YTHDF proteins may be administered in an amount, and/or using a vehicle (e.g., LNPs), that results in upregulation in one or more cell types, tissues, or organs, in the human subject (e.g., in a plurality of neurons in the brain of the human subject). Such constructs may be designed to include tissue or cell-type specific promoters. In some aspects, longevity may be improved by at least 5, 10, 15, 20, 25, 30, 35, 40, 45, or 50%, or by a percentage within a range with endpoints defined by any of the foregoing values. In some aspects, the nucleotide constructs or YTHDF proteins may be administered using any of the formulations, dosage amounts, or dosage regimens described herein. For example, mRNAs encoding at least one YTHDF protein or fragment described herein may be administered to a human subject (e.g., in any therapeutic composition described herein) in an amount of 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, 300-400 μg, 350-450 μg, or 400-500 μg per dose. In some aspects, a dosage selected from the list above is administered at least 1, 2, 3, 4, 5, 6, 7, 8, 9, 10, 11, or 12 times per day, week, month, or year. In some aspects, the dosage amount may vary over time (e.g., a plurality of dosage amounts may be independently selected from the list above and administered to the human subject according to any schedule listed above).

In some aspects, the disclosure provides use of a lipid nanoparticle, and an optional pharmaceutically acceptable carrier, in the manufacture of a medicament for treating or delaying progression of neurodegeneration, or for increasing longevity, in a human subject, wherein the medicament comprises the lipid nanoparticle and an optional pharmaceutically acceptable carrier and wherein the treatment comprises administration of the medicament to treat or delay progression of AD, ALS, FTD, or PD, or to increase longevity, according to any of the exemplary aspects described herein.

In some aspects, the disclosure provides a kit comprising a medicament comprising a lipid nanoparticle, and an optional pharmaceutically acceptable carrier, or a pharmaceutical composition, and a package insert comprising instructions for administration of the medicament alone or in combination the optional pharmaceutically acceptable carrier to treat or delay progression of AD, ALS, FTD, or PD, or to increase longevity, according to any of the exemplary aspects described herein.

In the interest of clarity not all of the routine features of the aspects are disclosed herein. It is understood that in the development of an actual implementation of the present disclosure, numerous decisions must be made in order to achieve specific goals (e.g., methods of treating specific neurodegenerative diseases), and that these specific goals will vary in different implementations. It will be appreciated that such a efforts might be complex and time-consuming, but would nevertheless be a routine undertaking for those of ordinary skill in the art, having the benefit of the present disclosure.

All publications and patents cited in this specification are herein incorporated by reference as if each individual publication or patent were specifically and individually indicated to be incorporated by reference in their entirety.

Furthermore, it is understood that the phraseology or terminology used herein is for the purpose of description and not of restriction, such that the terminology or phraseology of the present disclosure is to be interpreted in light of the teachings and guidance presented herein, in combination with knowledge available to a person of ordinary skill in the relevant art(s) at the time of invention. Moreover, it is not intended for any term in the specification or claims to be ascribed an uncommon or special meaning unless explicitly set forth as such in the specification.

The various aspects disclosed herein encompass present and future known equivalents to the structural and functional elements referred to herein by way of illustration. Moreover, while various aspects and applications have been shown and described herein, it will be apparent to those skilled in the art having the benefit of this disclosure that many more modifications than those mentioned above are possible without departing from the inventive concepts disclosed herein. For example, one of ordinary skill in the art would readily appreciate that individual features from any of the exemplary aspects disclosed herein may be combined to generate additional aspects that are in accordance with the inventive concepts disclosed herein.

It is further understood that any combination of elements or steps described herein may be used alone or in combination with still further unrecited elements or steps. To that end, any reference to the transitional phrase “comprising” recited herein is expressly understood to also include support for alternative aspects directed to a closed set (i.e., “consisting of” only the recited elements) and for a semi-closed set (i.e., “consisting essentially of” the recited elements and any additional elements or steps that do not materially affect the basic and novel characteristics of the invention).

Although illustrative exemplary aspects have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

SEQUENCE LISTING

SEQ ID NO: 1-D. melanogaster Ythdf Protein Sequence (700 aa)
>NP_651322.1 YTH N6-methyladenosine RNA binding protein, isoform
A [D. melanogaster]
MSGVDQMKIPGNTAKSVEERNIPWSQQVDEASYENLSSPTHDEVSGNDNSMQFQYPPFNFKENCNTPWNN
MNKGRKANANHDSYRRHGHSIPNNTRRDEHQVNPWNSRKPAAQSFRNENDQPSTKLDNRTSDEAQNQEVV
AAPKKTTWASIASQPAKLTSRAASTTSNSKKKGPGMPPPPMVPGKHNLDVNVWDLPNNKPPPVPSPPSPI
DLGYSDLSSDESISSNTAPPLGARAEKVHKDTENFLKYEHKGLGNNNNFSRAKVSPTGPVRRNLGPQQPV
HHAAPRPATNAGPFGPPNARRHDGGPHPSRNSERSGNYSSFRGSEFESASKFEYRDENQSRPVEATSATE
ELPVDSQLVLDELKDKNNYNPKVLDLKKAGSARFFVIKSYSEDDIHRSIKYEIWCSTDHGNKRLDDAFKE
RHEEGGNIMLFFSVNGSGHFCGMAQMMTPVDYNSTSSVWSQDKWRGKFKVKWIYVKDVPNGTLRHIRLEN
NENKSVTNSRDTQEVPNDKGIEVLQILHSYNHSTSIFDDFFHYEKKQEEEVSSKRPPMHGPDGNNHAPAP
LARSENRQADDKDRDRDGRGGMSQKHENSGGGAGERNPGHRGGAGSGGSHNNYSEYRRGEDIHKSSSDYQ
FKDRDGPDFDRENDEGSHYGSNNRKNEYKHNNTNKARLKTRDRDESTEQIKIGVRFKDTDKDKMRSNEYS
SEQ ID NO: 2-H. sapiens YTHDF1 Protein Sequence (559 aa)
>NP_060268.2 YTH domain-containing family protein 1 [H. sapiens]
MSATSVDTQRTKGQDNKVQNGSLHQKDTVHDNDFEPYLTGQSNQSNSYPSMSDPYLSSYYPPSIGFPYSL
NEAPWSTAGDPPIPYLTTYGQLSNGDHHEMHDAVEGQPGGLGNNIYQHRENFFPENPAFSAWGTSGSQGQ
QTQSSAYGSSYTYPPSSLGGTVVDGQPGFHSDTLSKAPGMNSLEQGMVGLKIGDVSSSAVKTVGSVVSSV
ALTGVLSGNGGTNVNMPVSKPTSWAAIASKPAKPQPKMKTKSGPVMGGGLPPPPIKHNMDIGTWDNKGPV
PKAPVPQQAPSPQAAPQPQQVAQPLPAQPPALAQPQYQSPQQPPQTRWVAPRNRNAAFGQSGGAGSDSNS
PGNVQPNSAPSVESHPVLEKLKAAHSYNPKEFEWNLKSGRVFIIKSYSEDDIHRSIKYSIWCSTEHGNKR
LDSAFRCMSSKGPVYLLFSVNGSGHFCGVAEMKSPVDYGTSAGVWSQDKWKGKEDVQWIFVKDVPNNQLR
HIRLENNDNKPVTNSRDTQEVPLEKAKQVLKIISSYKHTTSIFDDFAHYEKRQEEEEVVRKERQSRNKQ
SEQ ID NO: 3-H. sapiens YTHDF2 Protein Sequence (579 aa)
>NP_057342.2 YTH domain-containing family protein 2 isoform 1
[H. sapiens]
MSASSLLEQRPKGQGNKVQNGSVHQKDGLNDDDFEPYLSPQARPNNAYTAMSDSYLPSYYSPSIGFSYSL
GEAAWSTGGDTAMPYLTSYGQLSNGEPHFLPDAMFGQPGALGSTPFLGQHGENFFPSGIDFSAWGNNSSQ
GQSTQSSGYSSNYAYAPSSLGGAMIDGQSAFANETLNKAPGMNTIDQGMAALKLGSTEVASNVPKVVGSA
VGSGSITSNIVASNSLPPATIAPPKPASWADIASKPAKQQPKLKTKNGIAGSSLPPPPIKHNMDIGTWDN
KGPVAKAPSQALVQNIGQPTQGSPQPVGQQANNSPPVAQASVGQQTQPLPPPPPQPAQLSVQQQAAQPTR
WVAPRNRGSGFGHNGVDGNGVGQSQAGSGSTPSEPHPVLEKLRSINNYNPKDEDWNLKHGRVEIIKSYSE
DDIHRSIKYNIWCSTEHGNKRLDAAYRSMNGKGPVYLLFSVNGSGHFCGVAEMKSAVDYNTCAGVWSQDK
WKGRFDVRWIFVKDVPNSQLRHIRLENNENKPVTNSRDTQEVPLEKAKQVLKIIASYKHTTSIFDDFSHY
EKRQEEEESVKKERQGRGK
SEQ ID NO: 4-H. sapiens YTHDF3 Protein Sequence (585 aa)
>NP_689971.4 YTH domain-containing family protein 3 isoform a
[H. sapiens]
MSATSVDQRPKGQGNKVSVQNGSIHQKDAVNDDDFEPYLSSQTNQSNSYPPMSDPYMPSYYAPSIGFPYS
LGEAAWSTAGDQPMPYLTTYGQMSNGEHHYIPDGVFSQPGALGNTPPFLGQHGFNFFPGNADFSTWGTSG
SQGQSTQSSAYSSSYGYPPSSLGRAITDGQAGFGNDTLSKVPGISSIEQGMTGLKIGGDLTAAVTKTVGT
ALSSSGMTSIATNSVPPVSSAAPKPTSWAAIARKPAKPQPKLKPKGNVGIGGSAVPPPPIKHNMNIGTWD
EKGSVVKAPPTQPVLPPQTIIQQPQPLIQPPPLVQSQLPQQQPQPPQPQQQQGPQPQAQPHQVQPQQQQL
QNRWVAPRNRGAGFNQNNGAGSENFGLGVVPVSASPSSVEVHPVLEKLKAINNYNPKDFDWNLKNGRVFI
IKSYSEDDIHRSIKYSIWCSTEHGNKRLDAAYRSLNGKGPLYLLFSVNGSGHFCGVAEMKSVVDYNAYAG
VWSQDKWKGKFEVKWIFVKDVPNNQLRHIRLENNDNKPVTNSRDTQEVPLEKAKQVLKIIATFKHTTSIF
DDFAHYEKRQEEEEAMRRERNRNKQ
SEQ ID NO: 5-H. sapiens YTHDF1 mRNA Sequence
>NM_017798.4 Homo sapiens YTH N6-methyladenosine RNA binding
protein 1 (YTHDF1), mRNA
GTCGCCGCCGCCGCCGCCATTGGAGTCGACGCCTCCTCAGTGCGTCCGCGTCCCGGGCTCACCGCCGCTG
CCGCCTCGCCAGGGGCCCGCGCGCCCAGCAGCCGCCGCCGCCGCCCGGCCGGCGCCCGGGGAATTGGCGG
CGGGGCCCGGGGCCGCGCGAGCTAGGGTGACAGGCCCGGCCTCTAGGGGAGGCCCGAGCCGGCGGGCGCC
CCGGCCCCGCGTCTAGTTGTTCATGAAGCATGTCGGCCACCAGCGTGGACACCCAGAGAACAAAAGGACA
AGATAATAAAGTACAAAATGGTTCGTTACATCAGAAGGATACAGTTCATGACAATGACTTTGAGCCCTAC
CTTACTGGACAGTCAAATCAGAGTAACAGTTACCCCTCAATGAGCGACCCCTACCTGTCCAGCTATTACC
CGCCGTCCATTGGATTTCCTTACTCCCTCAATGAGGCTCCGTGGTCTACTGCAGGGGACCCTCCGATTCC
ATACCTCACCACCTACGGACAGCTCAGTAACGGAGACCATCATTTTATGCACGATGCTGTTTTTGGGCAG
CCTGGGGGCCTGGGGAACAACATCTATCAGCACAGGTTCAATTTTTTCCCTGAAAACCCTGCGTTCTCAG
CATGGGGGACAAGTGGGTCTCAAGGTCAGCAGACCCAGAGCTCCGCGTATGGGAGCAGCTACACCTACCC
CCCGAGCTCCCTGGGTGGCACGGTGGTTGATGGGCAGCCAGGCTTTCACAGCGACACCCTCAGCAAGGCC
CCCGGGATGAACAGCCTGGAGCAGGGCATGGTTGGCCTGAAGATTGGGGACGTCAGCTCCTCCGCCGTCA
AGACGGTGGGCTCTGTCGTCAGCAGCGTGGCACTGACTGGTGTCCTTTCTGGCAACGGTGGGACAAATGT
GAACATGCCAGTTTCAAAGCCGACCTCGTGGGCTGCCATTGCCAGCAAGCCTGCAAAACCACAGCCTAAA
ATGAAAACAAAGAGCGGGCCTGTCATGGGGGGTGGGCTGCCCCCTCCACCCATAAAGCATAACATGGACA
TTGGCACCTGGGATAACAAGGGGCCTGTGCCGAAGGCCCCAGTCCCCCAGCAGGCACCCTCTCCACAGGC
TGCCCCACAGCCCCAGCAGGTGGCTCAGCCTCTCCCAGCACAGCCCCCAGCTTTGGCTCAACCGCAGTAT
CAGAGCCCTCAGCAGCCACCCCAGACCCGCTGGGTTGCCCCACGCAACAGAAACGCGGCGTTTGGGCAGA
GCGGAGGGGCTGGCAGCGATAGCAACTCTCCTGGAAACGTCCAGCCTAATTCTGCCCCCAGCGTCGAATC
CCACCCCGTCCTTGAAAAACTGAAGGCTGCTCACAGCTACAACCCGAAAGAGTTTGAGTGGAATCTGAAA
AGCGGGCGTGTGTTCATCATCAAGAGCTACTCTGAGGACGACATCCACCGCTCCATTAAGTACTCCATCT
GGTGTAGCACAGAGCACGGCAACAAGCGCCTGGACAGCGCCTTCCGCTGCATGAGCAGCAAGGGGCCCGT
CTACCTGCTCTTCAGCGTCAATGGGAGTGGGCATTTTTGTGGGGTGGCCGAGATGAAGTCCCCCGTGGAC
TACGGCACCAGTGCCGGGGTCTGGTCTCAGGACAAGTGGAAGGGGAAGTTTGATGTCCAGTGGATTTTTG
TTAAGGATGTACCCAATAACCAGCTCCGGCACATCAGGCTGGAGAATAACGACAACAAACCGGTCACAAA
CTCCCGGGACACCCAGGAGGTGCCCTTAGAAAAAGCCAAGCAAGTGCTGAAAATTATCAGTTCCTACAAG
CACACAACCTCCATCTTCGACGACTTTGCTCACTACGAGAAGCGCCAGGAGGAGGAGGAGGTGGTGCGCA
AGGAACGGCAGAGTCGAAACAAACAATGAGGGCGAACCAGTTTCTTACATGTTCTAACGTTTGACTTTGA
AAACAGTTTAAAACACGTGTGCTTGGTCAGCTCCAGTGTGTCGTCCCGTGCGGGGGTTGAGTGTTGCATC
TTTGCCTTTCTTGTCGTTGATTTTTGCCCAGATGGATCTGCATTTATTTGTACTTTTTCTATGTATTATA
ATCCTGTAGAAGTCACTAATAAAGGAGTATTTTTTTTGTCAGCTTATCAATCAGACTGATCTAATGTGAA
ATGTAAGTATCCTTAAAAACAAAGCATCTATTTTGGCAGAAATTGTGTTCTTAAATTCAGTCATTTGATA
TTCTGTGAGACTTCATATTTCTCATCCCTTTATTGCTTTTTAGCAAACATAAGAAACCATGAGTCATTTT
GTCATTTAGAGTATTCTGATAAAATCTCTTGAAAATACTGAAATCAAAAGGTTAATGATTTTTTGTTCAT
TCTGATTTGTCATTTTATTATCTGTTATCGGTCTAAAGTGCTAATTTACCCATTTGATTTTTCTGCTAGA
CAGATAACTTTTAATTTTTCAAATTTGGCAGACACTTTTTTTTTTTTTTTGAAAATCTTTCCTTCCAGAT
CTGTTGCCCACTGAACAGCCACCCGTCCCTCACTGTCCTGGTGTCCGATTGGGCTGGATGGTGTTGGGGC
ATGATGTGTGGAGGAACTGGAAGGTGCTTTAGGTCTGGTTCAGGGTCGGGCATTCTTTGTTGTTTGCACA
TCTTTTTAAATTTTACACCTTTTCTTAAGAATTCTAATGCCGTCTTAAGTTTTTATACCAATAATGCTGA
GCTTTAAGTGTAGGATCTGGTAGTACAGACAGTGTGATGGATGATGCTGCTGGTTGTAAATTTCATCGTG
TGTGTCTAATTTTTTTTCCTGTTGAATGGGTAAAAACAAAACAAAACTTTTTTTAGAAGATGAATTTGCT
GTCATGTTTTGTGGAATGAGGGACCGTTGAGCTCACTACCACCTGGAGTTTGAGTTGAAGCATGAAAATG
GTGCCCATGCCTGACGCTCCAGCGCCTGGATCTGCACGTGCCCTTGTAGAGGATCCTTACCGTCCTAGAG
AGCAGACGCTTTCTGAAAACTACTTGCTCCAAAAGACCCTCTGAGTTAACGTTTCAGCTGTATCATTAGA
CTTGTATTTAGAGCGTGTCACTTCCTCTGAACTGTTACTGCCTGAATGGAGTCCTGGACGACATTGGGTT
TTTCCTCTAGGAGAATACAAGCCTTAATAAACAATACTATTTAGCAAA
SEQ ID NO: 6-H. sapiens YTHDF2 mRNA Sequence
>NM_016258.3 Homo sapiens YTH N6-methyladenosine RNA binding
protein 2 (YTHDF2), mRNA
AGAGCGTCGCCGAGTCGGAGCCGGAGCCTGAGCCGCGCGCTGTGTCTCCGCTGCGTCCGCCGAGGCCCCC
GAGTGTCAGGGACAAAAGCCTCCGCCTGCTCCCGCAGACGGGGCTCATCTGCCGCCGCCGCCGCGCTGAG
GAGAGTTCGCCGCCGTCGCCGCCCGTGAGGATCTGAGAGCCATGTCGGCCAGCAGCCTCTTGGAGCAGAG
ACCAAAAGGTCAAGGAAACAAAGTACAAAATGGATCTGTACATCAAAAGGATGGATTAAACGATGATGAT
TTTGAACCTTACTTGAGTCCACAGGCAAGGCCCAATAATGCATATACTGCCATGTCAGATTCCTACTTAC
CCAGTTACTACAGTCCCTCCATTGGCTTCTCCTATTCTTTGGGTGAAGCTGCTTGGTCTACGGGGGGTGA
CACAGCCATGCCCTACTTAACTTCTTATGGACAGCTGAGCAACGGAGAGCCCCACTTCCTACCAGATGCA
ATGTTTGGGCAACCAGGAGCCCTAGGTAGCACTCCATTTCTTGGTCAGCATGGTTTTAATTTCTTTCCCA
GTGGGATTGACTTCTCAGCATGGGGAAATAACAGTTCTCAGGGACAGTCTACTCAGAGCTCTGGATATAG
TAGCAATTATGCTTATGCACCTAGCTCCTTAGGTGGAGCCATGATTGATGGACAGTCAGCTTTTGCCAAT
GAGACCCTCAATAAGGCTCCTGGCATGAATACTATAGACCAAGGGATGGCAGCACTGAAGTTGGGTAGCA
CAGAAGTTGCAAGCAATGTTCCAAAAGTTGTAGGTTCTGCTGTTGGTAGCGGGTCCATTACTAGTAACAT
CGTGGCTTCCAATAGTTTGCCTCCAGCCACCATTGCTCCTCCAAAACCAGCATCTTGGGCTGATATTGCT
AGCAAGCCTGCAAAACAGCAACCTAAACTGAAGACCAAGAATGGCATTGCAGGGTCAAGTCTTCCGCCAC
CCCCGATAAAGCATAACATGGATATTGGAACTTGGGATAACAAGGGTCCCGTTGCAAAAGCCCCCTCACA
GGCTTTGGTTCAGAATATAGGTCAGCCAACCCAGGGGTCTCCTCAGCCTGTAGGTCAGCAGGCTAACAAT
AGCCCACCAGTGGCTCAGGCATCAGTAGGGCAACAGACACAGCCATTGCCTCCACCTCCACCACAGCCTG
CCCAGCTTTCAGTCCAGCAACAGGCAGCTCAGCCAACCCGCTGGGTAGCACCTCGGAACCGTGGCAGTGG
GTTCGGTCATAATGGGGTGGATGGTAATGGAGTAGGACAGTCTCAGGCTGGTTCTGGATCTACTCCTTCA
GAACCCCACCCAGTGTTGGAGAAGCTTCGGTCCATTAATAACTATAACCCCAAAGATTTTGACTGGAATC
TGAAACATGGCCGGGTTTTCATCATTAAGAGCTACTCTGAGGACGATATTCACCGTTCCATTAAGTATAA
TATTTGGTGCAGCACAGAGCATGGTAACAAGAGACTGGATGCTGCTTATCGTTCCATGAACGGGAAAGGC
CCCGTTTACTTACTTTTCAGTGTCAACGGCAGTGGACACTTCTGTGGCGTGGCAGAAATGAAATCTGCTG
TGGACTACAACACATGTGCAGGTGTGTGGTCCCAGGACAAATGGAAGGGTCGTTTTGATGTCAGGTGGAT
TTTTGTGAAGGACGTTCCCAATAGCCAACTGCGACACATTCGCCTAGAGAACAACGAGAATAAACCAGTG
ACCAACTCTAGGGACACTCAGGAAGTGCCTCTGGAAAAGGCTAAGCAGGTGTTGAAAATTATAGCCAGCT
ACAAGCACACCACTTCCATTTTTGATGACTTCTCACACTATGAGAAACGCCAAGAGGAAGAAGAAAGTGT
TAAAAAGGAACGTCAAGGTCGTGGGAAATAAAAGGCAGTTCTACACAGACTGCAGCAACGGTTGCATCTG
CATATCCTAAGAGGAAAAAATGACCTTCAAGAGAATTAGGACTTTTTTCTTAATTTCACTGACTTCAGAG
ACGATTGCAGACTTGCAGTTTAAGTATTGGAATTTCACAAAAGACATAGGACTTAACTGGAAAATGAAAA
AAAAAAGAAAAAGAAAAAACTAAACAAAAAATCCCTCTAGGTAGTTTAGGTGAAAAATGTCCCTTTTATT
TTGGCTTTGGTTGTGATTTCAGAGCATAATGCTATGTTTTTTTGTCTTTTTACTATGTTTTTCGGATTTT
TAAGTCCGTAAGTGCATACAGTTTTCTCTAATTTTTAAACCCTTTCCTCCTCCCATTTTGACATTTGCAC
TTGGAGAACACTTGAGTTGTGAAGGTTTTGGGCATCCACCCCAGAAAGTGGGAATTTGATTTTATCCTTC
CGAACTGGAAGAACATTTTTATGAAGAATTTTTGTCTAGGAGAATATAACAGTGTTACCCAAGGTTGTGT
CTTTAAGGGTGGTTCATTTTCTCTGACCTTTTGTTACTCAAAGTAAAGTACTAGGAGTCCTAAGAAATGT
TCTGTTCTTGTACATTATACTGATTAAGTCAGGATTAATTTGATTTCAAAGCTGAGAACAGTGGTAAAAA
CTCGTTTACAGAAATGCATTTTGGAAGAGAAAAATACTGTAAAACGTGTCGTGAATGTTTCTTCAGTTTC
TTGTTCAGCCAATGAGGAAAGGGCATTGCCTTTCTTTTTACCATTAATCACTTCTCAATAAACGTGAGAT
CCTGTTGAGCATCA
SEQ ID NO: 7-H. sapiens YTHDF3 mRNA Sequence
>NM_152758.6 Homo sapiens YTH N6-methyladenosine RNA binding
protein 3 (YTHDF3), mRNA
GAGACTAGGGAGTCTGTCCGCCATTGTGGACCCGAGAAGCAGAGAGCGAGAGGGGGAAGAGGAGCGTGCA
AGCGGAAAAGACGGGCCTCTTCCTCCGACTCCCGAGCGCGAGGCCCTCATTTTGGGTTCTCAGCGAACGG
CGGCAGCGGCGGCGGCTGGAACAATCACTCGGCCAAGGGCGACAGCCAACTGCTGTGAGTGCACGGGGAG
AGGCCCAGGCAGCGGCGGCGGCGGCGGCTCTCGGGTTGCGGTGAAGAATGTCAGCCACTAGCGTGGATCA
GAGACCTAAAGGGCAAGGAAATAAAGTTTCAGTACAAAACGGTTCGATTCATCAAAAAGATGCTGTAAAT
GATGATGATTTTGAGCCATACTTAAGTAGCCAGACAAATCAGAGTAACAGCTATCCACCAATGTCAGATC
CATACATGCCTAGTTACTATGCTCCATCCATTGGATTTCCATATTCTCTTGGGGAAGCAGCGTGGTCCAC
AGCTGGAGACCAGCCTATGCCATATCTGACAACCTATGGACAAATGAGTAATGGAGAACATCACTATATA
CCAGATGGTGTATTTAGTCAACCTGGGGCATTAGGAAATACCCCTCCATTTCTTGGTCAACATGGATTTA
ACTTTTTTCCTGGTAATGCTGATTTCTCTACATGGGGGACAAGTGGATCTCAGGGACAATCAACACAAAG
TTCTGCTTATAGTAGCAGTTATGGCTATCCACCTAGTTCTCTTGGGAGAGCTATTACTGATGGACAGGCT
GGATTTGGCAATGATACTTTGAGTAAGGTGCCTGGCATTAGCAGTATTGAGCAAGGCATGACTGGACTGA
AAATTGGTGGTGACCTGACAGCTGCAGTGACAAAAACTGTAGGTACAGCTTTGAGCAGCAGTGGTATGAC
TAGCATTGCAACCAATAGTGTGCCCCCAGTTAGCAGTGCAGCACCTAAACCAACCTCCTGGGCTGCCATT
GCCAGAAAGCCTGCCAAACCTCAACCGAAACTTAAACCCAAGGGCAATGTGGGAATTGGGGGTTCTGCTG
TACCACCACCTCCTATAAAACACAACATGAATATTGGAACTTGGGATGAAAAAGGGTCAGTGGTAAAGGC
TCCACCAACCCAACCAGTTCTGCCTCCTCAAACTATAATCCAGCAGCCTCAGCCATTAATTCAACCACCA
CCATTGGTGCAAAGCCAACTGCCTCAACAGCAGCCTCAACCACCACAACCACAGCAGCAACAAGGACCTC
AGCCACAGGCCCAGCCTCACCAAGTGCAGCCTCAACAGCAGCAGCTGCAGAATCGCTGGGTAGCTCCTCG
TAACAGGGGAGCAGGCTTCAACCAGAACAATGGAGCGGGCAGTGAAAACTTTGGTTTAGGTGTTGTACCT
GTCAGTGCTTCACCTTCTAGTGTAGAAGTGCATCCCGTGCTGGAAAAGCTAAAGGCCATAAACAACTATA
ATCCCAAAGACTTTGATTGGAATCTGAAGAATGGACGTGTGTTTATAATTAAAAGCTACTCTGAGGATGA
CATACATCGTTCCATTAAATACTCTATCTGGTGTAGTACTGAGCATGGTAATAAGCGTTTGGATGCAGCT
TACCGTTCCCTGAATGGGAAAGGCCCACTCTATTTACTCTTCAGTGTGAATGGCAGTGGACATTTTTGTG
GAGTGGCTGAAATGAAGTCTGTTGTGGACTATAATGCGTATGCTGGTGTCTGGTCTCAGGATAAGTGGAA
GGGCAAATTTGAAGTTAAATGGATCTTTGTCAAAGATGTTCCCAATAACCAATTACGGCATATTCGCTTA
GAAAATAATGACAACAAACCGGTTACCAATTCAAGGGACACTCAAGAGGTACCCCTAGAAAAAGCTAAGC
AAGTGCTTAAAATAATTGCTACTTTCAAGCATACCACCTCAATCTTTGATGACTTTGCACATTATGAAAA
GCGTCAAGAAGAGGAGGAAGCCATGCGTAGGGAGAGAAATAGAAACAAACAATAACCGTATGAAGATGTC
CTGTTAAATTTACAACACTAACGATGTAGACTCTGGAAATGCCTAATAAGTCAAAGAAGACGTATTAAAG
CTCTTTTCTGCTTAAGGTGACATCTTTGAACACTTTAACACAAAGTTGACTCTTCTCGTAATGGTTTTCA
TCAGCGCATCTGCCCTTATACTCTTCACCAAACACACTTGAGAACTGTAACTTCGTCAAGCACTTTCTGT
CCTGAAGCTTTTACCAGTATCTGCTGTCTTTTGTAATTATGCATCCTAGCTAAGGCACAGAAGACTGAAT
GAATGCAAGGATTCATTAACTCTTTGAATTTGTTAAATACTAACAGTTAACCATTAGAAGTGGTTCAATG
ATGTAAGAGTCACACTGCTTCAACTTTTTCTTTGTTGTAGTTTTTAAATTGTCGATTTTTAGCTATTTGA
CAGATTAAAAGCAAAATAATCATGCCATATTTAGTCCTGGAGTTCAAGTCTAAATGTTGATGTGAAAAAT
TATTGTAGTAAACTTTTAATATGGCAAAGCAACCTTAAGCTCTATTTTAGCCAAATGAAACATAATCTGA
AATTATATTAGAACATTTCCCTTGTCTTCAAACTGTTTGGTGTAACAGAATATTGATATGCAGCTTGGTG
GATTTCACCAGTTAATGCACATTCTTCTTCCCTCCTCCCCCCATTAATATGTATACTGAAAAATGTGCAT
TTGTCTGAGGAATTATTTTGTTTGCTACCACTTAATGAATCTCAAAATTTTGAGTAAATGTACCTCAGTC
TAATCAGACTTTTTATGACCTTTATAACTACATTTAAAACCCTTAATTCCTATTTCTGGGTGTTTGCGAG
CCTGATTGCTATCATGAAGTAAAAATTTATTACTCTAGGTATTCACTAGCTAAATAAACATAGTTCTTGT
TTAGCAAGCATATGTTGTTCCTCAGCTCTTTTCTCCAGCTTTTGCAGTGTCCTGGCATCCTTAAAATACT
TTGAAAATATGGCCTTGATCCATGGATTAAATCAGTATCTAAGTGAATGTGTTGATGTTTTATTGATCAG
ATCTATATAAGTGGGAATACAGCATATATCTGGATATTCTTATAGTTATCTTTTTAACATCTTATTTTTT
TCATTAATTACATATCAACATTAATTTTGTATCTTGAAGCAAATTGATTTTGTATAATTAAATGTGTCAA
GCATCTGTATTAATTGATTTGATGGCATAAGGTTATGAAAATAATGTACTGCCCCATGTATTACTGTTCC
AAAAGGAGAAAGCTATGTAGAAAGATACATTAAGGGTGAAAATAGCAATACAGTAGATTTGAATACCTTG
ATGTTTTGCATTACTTCATTTATGTTTACATCATGTTTAGAAATGTTTTCATTTACTGTGGTCTTTGGTC
ACTTCAGCTCAAAGACCTAGTGATGGATATTTCTTTGAGGCTTTCATTTATATAATTTTATTTTGTACAA
TGTTTTTTTTAAATGTGCAAATACTGTATTCAAGTGAAAAAAATACAGTATTTGTAGATAACCATAGCTA
CTACACAGTTCTTCGGTAGTCCCAGTGTAGTTATATCAGTGTTTACTGAAGGGAACATCAAAATATTAAT
GGTATATTATAAAATAAAGACTTTCTTAAAGGAAAATTGCACCTATTTTACCTTTTTAAGAGTAAGCCAT
GAAATCTTGTAACATGTCTCTTAACTATTTATAATGAAAAGTGGCATTTGGGTATAGTCACCACAGCAAT
GTTCTACATCCCTAAGATTATCTAGGTAGGACATGTCAAAGATGACTGTTGTCATTCTGGAGGTCCTATT
AGAGAATATTATAAAAGGGTGACCTTGTAGGAAGGATCTGAGTCCTCCCCCTGAGGTTCTCTTTTTCTTG
GTGCTTTATTAGCAACTCTGGATATTTTTATAAAACTAGTTACATTATAAACGGTTTCAAACATGTTTAA
TTTACATTAGGTTTTTATGTAAGAGTGTCATGGAAGCACTCAGCAAGCAGGCTGATTGCAATAGACTCAG
ACATGCGAATAAATGTAATTGAGAGTCTATTCATGGTGAGGAGTACATCCCAGTGCCTTTAACCTGGATT
TCTAATCTTAAGTGAAATGGGTGCAGCATTCCTTTGGAAAAAAAAATCTTTTTATTTTCAAGTGATAATT
TTGTGTTTTTCTCATATAAGTTTTCTCCAGAGCACCCACCTTCTCTTCCTTCTTGGTCTGTCATTATATT
GCAAAATATTTTTCCTCTGAATGAAATTATCACAGGTTGTCTCAAGCACAACCAACTGAATGTCTCTTAA
CTGTGGGGACCAAAAGGGAGAGAGCCTGGGGTCTACAAGAGGAGACACATCATCAAATGTTTGAATGATC
ACAAATTAAGACATTATCAGCCCAGTAAATTTCTTGCTTAATGTTTTTCCAAGTTCTGGCTTGAATATTT
CTTATTAAAGCTATCTTATGTGGGTATTTTATTTTGAAAGGTATTATAGTTTGTATATTTAACAGTAAGG
AGGAAACTGTAACCAAAATTAGTATTTCTCTATACGTATTGGTACTTGAAGATTCCTTTCAAAAGAAATC
CAGCGTTTTCCTAATTTTAGTACTTAATTTCTCTTTTTAATTTAAGTGATCTTTCTAATTCGAAAGCTGT
GTTCTTTTTGAATACCGTGCATGGGGGTTAAGCTGATGTTAAAACAGTTTGCAATAAAAAAAAATGAATC
AGCTTAAGTCATTTAATCATTTCAAGTGCATTCTGCATCCTTTAAAAATAAGTTTAAGAAATTTAAGAGA
ATTGTGTTTTCATTAAGTTTTGCATATCTTTTGTTATGCCATGTAAATTCCCTTTTTCGTATGATTAAAG
GAAGGTTATGATAAAATGATTAGTTCATTTACATTCACTTGTAGCAATTACATGAGAATTTGAATTTTGT
CGTGTTTGGGTTTGTTCATTCCTGTGAATGATGGTACAGTTAGGTGAGATTTTCTGTTATGGTACCCAAA
CTCACCATTTGGTCCTCTTTAATCTTTGAGGGTTTCAATAAAAATTGTTCACTCA
SEQ ID NO: 8-H. sapiens YTHDF1 (Reverse Translation)
>Reverse translation of NP_060268.2 YTHDF1 [H. sapiens].
augagcgcgaccagcguggaucagcgcccgaaaggccagggcaacaaagugagcgugcag
aacggcagcauucaucagaaagaugcggugaacgaugaugauuuugaaccguaucugagc
agccagaccaaccagagcaacagcuauccgccgaugagcgauccguauaugccgagcuau
uaugcgccgagcauuggcuuuccguauagccugggcgaagcggcguggagcaccgcgggc
gaucagccgaugccguaucugaccaccuauggccagaugagcaacggcgaacaucauuau
auuccggauggcguguuuagccagccgggcgcgcugggcaacaccccgccguuucugggc
cagcauggcuuuaacuuuuuuccgggcaacgcggauuuuagcaccuggggcaccagcggc
agccagggccagagcacccagagcagcgcguauagcagcagcuauggcuauccgccgagc
agccugggccgcgcgauuaccgauggccaggcgggcuuuggcaacgauacccugagcaaa
gugccgggcauuagcagcauugaacagggcaugaccggccugaaaauuggcggcgaucug
accgcggcggugaccaaaaccgugggcaccgcgcugagcagcagcggcaugaccagcauu
gcgaccaacagcgugccgccggugagcagcgcggcgccgaaaccgaccagcugggcggcg
auugcgcgcaaaccggcgaaaccgcagccgaaacugaaaccgaaaggcaacgugggcauu
ggcggcagcgcggugccgccgccgccgauuaaacauaacaugaacauuggcaccugggau
gaaaaaggcagcguggugaaagcgccgccgacccagccggugcugccgccgcagaccauu
auucagcagccgcagccgcugauucagccgccgccgcuggugcagagccagcugccgcag
cagcagccgcagccgccgcagccgcagcagcagcagggcccgcagccgcaggcgcagccg
caucaggugcagccgcagcagcagcagcugcagaaccgcuggguggcgccgcgcaaccgc
ggcgcgggcuuuaaccagaacaacggcgcgggcagcgaaaacuuuggccugggcguggug
ccggugagcgcgagcccgagcagcguggaagugcauccggugcuggaaaaacugaaagcg
auuaacaacuauaacccgaaagauuuugauuggaaccugaaaaacggccgcguguuuauu
auuaaaagcuauagcgaagaugauauucaucgcagcauuaaauauagcauuuggugcagc
accgaacauggcaacaaacgccuggaugcggcguaucgcagccugaacggcaaaggcccg
cuguaucugcuguuuagcgugaacggcagcggccauuuuugcggcguggcggaaaugaaa
agcgugguggauuauaacgcguaugcgggcguguggagccaggauaaauggaaaggcaaa
uuugaagugaaauggauuuuugugaaagaugugccgaacaaccagcugcgccauauucgc
cuggaaaacaacgauaacaaaccggugaccaacagccgcgauacccaggaagugccgcug
gaaaaagcgaaacaggugcugaaaauuauugcgaccuuuaaacauaccaccagcauuuuu
gaugauuuugcgcauuaugaaaaacgccaggaagaagaagaagcgaugcgccgcgaacgc
aaccgcaacaaacag
SEQ ID NO: 9-H. sapiens YTHDF2 (Reverse Translation)
>Reverse translation of NP_057342.2 YTHDF2 [H. sapiens].
augagcgcgagcagccugcuggaacagcgcccgaaaggccagggcaacaaagugcagaac
ggcagcgugcaucagaaagauggccugaacgaugaugauuuugaaccguaucugagcccg
caggcgcgcccgaacaacgcguauaccgcgaugagcgauagcuaucugccgagcuauuau
agcccgagcauuggcuuuagcuauagccugggcgaagcggcguggagcaccggcggcgau
accgcgaugccguaucugaccagcuauggccagcugagcaacggcgaaccgcauuuucug
ccggaugcgauguuuggccagccgggcgcgcugggcagcaccccguuucugggccagcau
ggcuuuaacuuuuuuccgagcggcauugauuuuagcgcguggggcaacaacagcagccag
ggccagagcacccagagcagcggcuauagcagcaacuaugcguaugcgccgagcagccug
ggcggcgcgaugauugauggccagagcgcguuugcgaacgaaacccugaacaaagcgccg
ggcaugaacaccauugaucagggcauggcggcgcugaaacugggcagcaccgaaguggcg
agcaacgugccgaaaguggugggcagcgcggugggcagcggcagcauuaccagcaacauu
guggcgagcaacagccugccgccggcgaccauugcgccgccgaaaccggcgagcugggcg
gauauugcgagcaaaccggcgaaacagcagccgaaacugaaaaccaaaaacggcauugcg
ggcagcagccugccgccgccgccgauuaaacauaacauggauauuggcaccugggauaac
aaaggcccgguggcgaaagcgccgagccaggcgcuggugcagaacauuggccagccgacc
cagggcagcccgcagccggugggccagcaggcgaacaacagcccgccgguggcgcaggcg
agcgugggccagcagacccagccgcugccgccgccgccgccgcagccggcgcagcugagc
gugcagcagcaggcggcgcagccgacccgcuggguggcgccgcgcaaccgcggcagcggc
uuuggccauaacggcguggauggcaacggcgugggccagagccaggcgggcagcggcagc
accccgagcgaaccgcauccggugcuggaaaaacugcgcagcauuaacaacuauaacccg
aaagauuuugauuggaaccugaaacauggccgcguguuuauuauuaaaagcuauagcgaa
gaugauauucaucgcagcauuaaauauaacauuuggugcagcaccgaacauggcaacaaa
cgccuggaugcggcguaucgcagcaugaacggcaaaggcccgguguaucugcuguuuagc
gugaacggcagcggccauuuuugcggcguggcggaaaugaaaagcgcgguggauuauaac
accugcgcgggcguguggagccaggauaaauggaaaggccgcuuugaugugcgcuggauu
uuugugaaagaugugccgaacagccagcugcgccauauucgccuggaaaacaacgaaaac
aaaccggugaccaacagccgcgauacccaggaagugccgcuggaaaaagcgaaacaggug
cugaaaauuauugcgagcuauaaacauaccaccagcauuuuugaugauuuuagccauuau
gaaaaacgccaggaagaagaagaaagcgugaaaaaagaacgccagggccgcggcaaa
SEQ ID NO: 10-H. sapiens YTHDF3 (Reverse Translation)
>Reverse translation of NP_689971.4 YTHDF3 [H. sapiens].
augagcgcgaccagcguggaucagcgcccgaaaggccagggcaacaaagugagcgugcag
aacggcagcauucaucagaaagaugcggugaacgaugaugauuuugaaccguaucugagc
agccagaccaaccagagcaacagcuauccgccgaugagcgauccguauaugccgagcuau
uaugcgccgagcauuggcuuuccguauagccugggcgaagcggcguggagcaccgcgggc
gaucagccgaugccguaucugaccaccuauggccagaugagcaacggcgaacaucauuau
auuccggauggcguguuuagccagccgggcgcgcugggcaacaccccgccguuucugggc
cagcauggcuuuaacuuuuuuccgggcaacgcggauuuuagcaccuggggcaccagcggc
agccagggccagagcacccagagcagcgcguauagcagcagcuauggcuauccgccgagc
agccugggccgcgcgauuaccgauggccaggcgggcuuuggcaacgauacccugagcaaa
gugccgggcauuagcagcauugaacagggcaugaccggccugaaaauuggcggcgaucug
accgcggcggugaccaaaaccgugggcaccgcgcugagcagcagcggcaugaccagcauu
gcgaccaacagcgugccgccggugagcagcgcggcgccgaaaccgaccagcugggcggcg
auugcgcgcaaaccggcgaaaccgcagccgaaacugaaaccgaaaggcaacgugggcauu
ggcggcagcgcggugccgccgccgccgauuaaacauaacaugaacauuggcaccugggau
gaaaaaggcagcguggugaaagcgccgccgacccagccggugcugccgccgcagaccauu
auucagcagccgcagccgcugauucagccgccgccgcuggugcagagccagcugccgcag
cagcagccgcagccgccgcagccgcagcagcagcagggcccgcagccgcaggcgcagccg
caucaggugcagccgcagcagcagcagcugcagaaccgcuggguggcgccgcgcaaccgc
ggcgcgggcuuuaaccagaacaacggcgcgggcagcgaaaacuuuggccugggcguggug
ccggugagcgcgagcccgagcagcguggaagugcauccggugcuggaaaaacugaaagcg
auuaacaacuauaacccgaaagauuuugauuggaaccugaaaaacggccgcguguuuauu
auuaaaagcuauagcgaagaugauauucaucgcagcauuaaauauagcauuuggugcagc
accgaacauggcaacaaacgccuggaugcggcguaucgcagccugaacggcaaaggcccg
cuguaucugcuguuuagcgugaacggcagcggccauuuuugcggcguggcggaaaugaaa
agcgugguggauuauaacgcguaugcgggcguguggagccaggauaaauggaaaggcaaa
uuugaagugaaauggauuuuugugaaagaugugccgaacaaccagcugcgccauauucgc
cuggaaaacaacgauaacaaaccggugaccaacagccgcgauacccaggaagugccgcug
gaaaaagcgaaacaggugcugaaaauuauugcgaccuuuaaacauaccaccagcauuuuu
gaugauuuugcgcauuaugaaaaacgccaggaagaagaagaagcgaugcgccgcgaacgc
aaccgcaacaaacag

1. A nucleic acid molecule, encoding a YTHDF1, YHTDF2, or YTHDF3 protein, operably linked to a heterologous promoter and/or an upstream activation sequence (UAS).

2. The nucleic acid molecule of claim 1, wherein:
the YTHDF1 protein has an amino acid sequence of SEQ ID NO: 2, or is encoded by a nucleotide sequence of SEQ ID NOs: 5 or 8;
the YTHDF2 protein has an amino acid sequence of SEQ ID NO: 3, or is encoded by a nucleotide sequence of SEQ ID NOs: 6 or 9; and
the YTHDF3 protein has an amino acid sequence of SEQ ID NO: 4, or is encoded by a nucleotide sequence of SEQ ID NOs: 7 or 10.

3. The nucleic acid molecule of claim 1, wherein:
the YTHDF1 protein has an amino acid sequence that is at least 95% identical to the sequence of SEQ ID NO: 2, or is encoded by a nucleotide sequence that is at least 95% identical to SEQ ID NOs: 5 or 8;
the YTHDF2 protein has an amino acid sequence that is at least 95% identical to the sequence of SEQ ID NO: 3, or is encoded by a nucleotide sequence that is at least 95% identical to SEQ ID NOs: 6 or 9; and
the YTHDF3 protein has an amino acid sequence that is at least 95% identical to the sequence of SEQ ID NO: 4, or is encoded by a nucleotide sequence that is at least 95% identical to SEQ ID NOs: 7 or 10;
wherein the YTHDF1, YTHDF2, or YTHDF3 protein is capable of specifically binding to N6-methyladenosine (m⁶A)-modified mRNA, when expressed in a human cell.

4. The nucleic acid molecule of claim 2, wherein the nucleic acid molecule is incorporated into:
a) a plasmid; or
b) a viral vector capable of transfecting a eukaryotic cell.

5. The nucleic acid molecule of claim 4, wherein the viral vector is an adenovirus, an adeno-associated virus, a retrovirus, and/or a lentivirus.

6. The nucleic acid molecule of claim 1, wherein the promoter is a cell-type specific promoter configured to promote expression of the nucleic acid molecule in a neuron, a glial cell, an astrocyte, a microglial cell, or an oligodendrocyte.

7. The nucleic acid molecule of claim 1, wherein the promoter is a neuron-specific promoter, selected from the group consisting of a synapsin I promoter, a calcium/calmodulin-dependent protein kinase II promoter, a tubulin alpha I promoter, a neuron-specific enolase promoter, and a platelet-derived growth factor beta chain promoter.

8. A eukaryotic cell, adapted to express the nucleic acid molecule of claim 1.

9. A method of treating a neurodegenerative disease in a subject in need thereof, comprising:
a) administering a therapeutically-effective amount of a YTHDF protein comprising
i) YTHDF1, YTHDF2, or YTHDF3; or
ii) a protein that is at least 95% identical to the sequence of SEQ ID NO: 2, 3, or 4, wherein the YTHDF protein is capable of specifically binding to N₆-methyladenosine (m⁶A)-modified mRNA, when expressed in a human cell; and
b) reducing or eliminating at least one symptom of the neurodegenerative disease.

10. The method of claim 9, wherein the therapeutically-effective amount comprises 0.01 to 5,000 mg/day.

11. The method of claim 9, wherein the neurodegenerative disease comprises: Alzheimer's disease (AD), amyotrophic lateral sclerosis (ALS), frontotemporal dementia (FTD), or Parkinson's disease (PD).

12. The method of claim 9, wherein the YTHDF protein is YTHDF1, YTHDF2, or YTHDF, and the therapeutically-effective amount comprises an amount sufficient to increase the level of endogenous YTHDF1, YTHDF2, or YTHDF3, in a cell of the subject, by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100%.

13. The method of claim 12, wherein the cell is a neuron, a glial cell, an astrocyte, a microglial cell, or an oligodendrocyte.

14. A method of treating a neurodegenerative disease in a subject in need thereof, comprising:
a) administering the nucleic acid molecule of claim 1 to the subject, under conditions sufficient to cause expression of the YTHDF1, YTHDF2, or YTHDF3 protein encoded by the nucleic acid, in a cell of the central nervous system of the subject; and
b) reducing or eliminating at least one symptom of the neurodegenerative disease;
wherein the nucleic acid molecule is administered in an amount of 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, 300-400, 350-450 μg, or 400-500 μg per dose;
optionally, wherein the nucleic acid is administered to the human subject at least a) once daily, b) 1, 2, 3, 4, 5, 6, or 7 times per week, or c) 1, 2, 3, or 4 times per month.

15. The method of claim 14, wherein the nucleic acid molecule is an mRNA or a DNA molecule.

16. The method of claim 14, wherein administering the nucleic molecule to the subject comprises:
a) administering a viral vector to the subject, wherein the viral vector encodes the nucleic acid molecule.

17. The method of claim 14, wherein the YTHDF1, YTHDF2, or YTHDF3 protein encoded by the nucleic acid molecule is expressed at a higher level compared to the level of endogenous expression of YTHDF1, YTHDF2, or YTHDF3, respectively, in the cell of the central nervous system of the subject.

18. The method of claim 14, wherein the nucleic acid molecule is administered in an amount sufficient to increase the level of endogenous YTHDF1, YTHDF2, or YTHDF3, in the cell of the central nervous system of the subject, by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30%.

19. The method of claim 18, wherein the cell is a neuron, a glial cell, an astrocyte, a microglial cell, or an oligodendrocyte.

20. A method of increasing the longevity of a human subject in need thereof, comprising:
a) administering the nucleic acid molecule of claim 1 to the subject, under conditions sufficient to cause expression of the YTHDF1, YTHDF2, or YTHDF3 protein encoded by the nucleic acid, in a cell of the central nervous system of the subject,
wherein the nucleic acid molecule is administered in an amount of 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, 300-400, 350-450 μg, or 400-500 μg per dose;
optionally, wherein the nucleic acid is administered to the human subject at least a) once daily, b) 1, 2, 3, 4, 5, 6, or 7 times per week, or c) 1, 2, 3, or 4 times per month.
